(19) United States
(12) Patent Application Publication
(10) Pub. No.: US 2023/0288433 A1
(43) Pub. Date: Sep. 14, 2023

(54) DETECTION OF PHOSPHOINOSITIDES IN BLOOD CELLS AS A BIOMARKER FOR ALPHA SYNUCLEIN ASSOCIATED PATHOLOGIES AND A METHOD OF TREATMENT OF PARKINSON'S DISEASE AND THE RELATED NEURODEGENERATIONS

(71) Applicant: YISSUM RESEARCH DEVELOPMENT COMPANY OF THE HEBREW UNIVERSITY OF JERUSALEM LTD., Jerusalem (IL)

(72) Inventors: Ronit SHARON, Mevasseret Zion (IL); Meir SCHECHTER, Kefar Adumim (IL); Suad ABD ALHADI, Nof Hagalil (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/077,711

(22) Filed: Dec. 8, 2022

Related U.S. Application Data
(63) Continuation-in-part of application No. PCT/IB2021/055806, filed on Jun. 29, 2021.

(51) Int. Cl. G01N 33/68

(52) U.S. Cl. CPC G01N 33/6896; G01N 2405/06; G01N 2800/2835; G01N 2800/52; G01N 2800/2821

(57) ABSTRACT

The present invention is directed to a method for determining a subject is afflicted with or having increased risk of developing an alpha-synuclein-associated disease or disorder, Parkinson's disease, or both. The method further includes administering to the subject determined as being afflicted with or having increased risk of developing an alpha-synuclein-associated disease or disorder a therapeutically effective amount of an agent characterized by being capable of decreasing signaling transduced by a member of PI4,5P₂ PI3,4P₂, PI3,5P2, PI3,4,5P₃, PI3P, PI4P signal transduction pathway.

### REFERENCE TO AN ELECTRONIC SEQUENCE LISTING

The contents of the electronic sequence listing (HUJI-P-076-CIP SQL_ST26_FINAL.xml; Size: 16,075 bytes; and Date of Creation: Dec. 8, 2022) is herein incorporated by reference in its entirety.

### CROSS-REFERENCE TO RELATED APPLICATIONS

This application is a Continuation-in-part of PCT Patent Application No. PCT/IB2021/055806 having International filing date of Jun. 29, 2021, the contents of which are incorporated herein by reference in their entirety.

### FIELD OF THE INVENTION

The present invention relates to Parkinson's disease therapy, including biomarkers for diagnosing same. Specifically, the invention concerns quantitative measurements of phosphoinositides in blood cells or samples, and use of pharmacological or genetic means, e.g., to maintain homeostasis of phosphoinositides in patients affected with PD.

### BACKGROUND

Parkinson disease (PD) is characterized by loss of neurons that reside in the substantia nigra pars compacta (SNc) and provide dopaminergic innervation to the dorsal striatum. A growing evidence now suggests that disease progression associates with axonal damage and synaptic loss in the striatum and only partly associates with the loss of dopaminergic neurons.

The cortex connects topologically with striatum through corticostriatal connections that play a central role in developing appropriate goal-directed behaviors, including motivation and cognition, to implement actions directed to obtain specific outcomes. Different regions of striatum have been associated with different cortical functions, including, emotions, cognition and motor control. Cortical glutamatergic efferents enter the striatum through striatal white matter tracts (WMTs), then make striatal synaptic contacts that influence the output nuclei of the basal ganglia.

α-Synuclein (α-Syn) is a protein critically implicated in the cytopathology and genetics of PD. In the brain, α-Syn pathology in the form of Lewy pathology, is strongly associated with disease progression and propagates in an ordered and predictable regional pattern. Neuroanatomy studies suggest that the length and caliber of axons, together with the degree of myelination, determine neuronal vulnerability to Lewy pathology. Specifically, susceptible cells are projection neurons that express α-Syn and generate long and thin axon, which is poorly or unmyelinated.

Compensatory mechanisms were suggested to take place at the pre-symptomatic stage of Parkinson's disease. According to this hypothesis, the surviving dopaminergic neurons go through functional changes aimed at preserving dopamine availability. It was further suggested that additional, non-dopaminergic mechanisms, are activated to support the changes in dopamine homeostasis. Experiments in animal models demonstrated compensatory, tyrosine hydroxylase (TH)-positive innervations, following acute, chemically-induced, lesion in SNc. Importantly, the compensating axonal branching associated with improvement in animal's motor performances. Depending on the size of the lesion, dopaminergic innervation was demonstrated in rodents and nonhuman primates.

The dopaminergic neurons of the SNc are characterized by a complex axonal arbor with an exceptionally high number of synapses in the striatum. This feature enables broad connectivity and neuroplasticity, and on the other hand, may underlie their specific vulnerability in PD, attributed to a high energetic burden. Collateral axonal branching is a multifaceted mechanism controlled by numerous factors. One such factor is Phosphatidylinositol 4,5-bisphosphate (PI4,5P2), is a phosphorylated derivative of the membrane phospholipid, phosphatidylinositol (PI). PI4,5P2 is generally formed through phosphorylation of PI4P by members of the type 1 PIP-5 kinase family (PIPKI). The three members of PIPKI family, PIPKI α, β and γ, were shown to play differential roles in neurite formation and elongation. Key proteins involved in mechanisms of axonal elongation and branching were shown to recruit members of PIPKI to this process. Interestingly, PIPKIα and PIPKIβ were shown to suppress the elongation of axons. However, PIPKIγ was implicated in axon elongation and organization of the growth cone. PI4P is arguably the most functionally diverse lipid molecule in eukaryotic cells. It is a precursor for the potent phosphoinositides, PI3,4P2, PI3,4,5P3 and PI4,5P2, each with its own array of cellular functions; it binds, recruits and/or activates many proteins involved in intracellular vesicular traffic; and plays a role in non-vesicular transfer of lipids. Lipid transfer between membrane compartments, in the form of non-vesicular lipid transfer, is essential for the maintenance of the unique lipid composition of biological membranes. Specific lipid transfer proteins are responsible for shuttling phospholipids and sterols that are synthesized in the ER to the PM, through ER-PM contact sites. Recent studies have demonstrated a role for PM PI4P as well as PI4,5P2 levels in lipid transfer. These studies also highlighted the importance of maintaining a gradient of PI4P concentrations, between cellular membranes, for lipid transfer through membrane contact site (MCS). Phosphatidylinositol 4-kinase type III a (PI4KA) synthesizes PI4P at the PM. Selective inhibitors for PI4KA were generated and shown to successfully lower PM PI4P, resulting in affected lipid exchange at MCS. PI4,5P2 levels were also lowered with the PI4KA inhibitor, however, in combination of PLC activation.

Phosphoinositide 3-kinases (PI3Ks) phosphorylate the 3-hydroxyl of the inositol ring of PI to generate PI3Ps. There are three classes of PI3K. Class IA and D3 PI3Ks preferentially phosphorylate PI4,5P2 to generate PI3,4,5P3. Class II α, β or γ PI3Ks phosphorylate PI or PI4P to PI3P or PI3,4P2, respectively. The sole class III PI3K isoform, Vps34, phosphorylates PI to PI3P and acts in concert with PD-associating genes. In accord, specific PIP phosphatases regulate PI3Ps levels. PTEN, a 3-phosphatase, removes the 3-phosphate from PI3,4,5P3 and PI3,4P2. Other 5- and 4-PIP-phosphatases further regulate PIP levels.

The serine/threonine kinase AKT, is recruited to cell membranes via direct binding to PI3,4,5P3 or PI3,4P2, where it is localized in the proximity of its two activating protein kinases, PDK1 and mTORC2 that phosphorylate AKT on Thr308 and Ser473, respectively. A proportional degree of AKT activation, generally depends on an equilibrium between synthesis by PI3Ks and hydrolysis by PTEN. The three closely related AKT isoforms are encoded by different genes, and share a high degree of homology. Data support different cellular functions for each of the AKT isoforms. Moreover, the AKT isoforms are differentially activated by PIPs. PIP3 selectively stimulates AKT1 and AKT3 at the plasma membrane (PM) and closely associates with Thr308 phosphorylation, PI3,4P2 activates AKT2 at both the PM and early endosomes and associates with Ser473. In accord, levels of PTEN protein modulate AKT Ser473 phosphorylation, and less so on Thr308. Importantly, the differential AKT phosphorylation reflects on its activity.

Regulation of PI3K activity by Ras palmitoylation: Protein S-palmitoylation involves the post-translational attachment of the 16-carbon FA palmitate to cysteine residues, that anchors the palmitoylated protein to cell membranes. A recent study has shown that α-Syn is involved in palmitoylation and accelerates Palmitate turnover on substrate proteins, whereas inhibition of acyl protein thioesterase 1 (APT1) a depalmitoylase, corrects an α-Syn-dependent palmitoylation deficit. Palmitoylation is critical for neuronal development and synaptic function. Members of class I and class II PI3Ks harbor a Ras-binding domain. Importantly, palmitoylation of Ras proteins (including Rab5 and Rab7) regulates PI3K activation. The palmitoyl acyltransferase (PAT) DHHC9-GCP16, was reported to palmitoylate Ras, whereas APT1 and ABHD17 proteins depalmitoylates H-Ras or N-Ras. The dynamic turnover of palmitoylation/depalmitoylation regulates the intracellular trafficking of HRas and NRas between the Golgi and cell membranes.

mTOR plays central roles in regulation of cell growth and metabolism. mTORC1 supports growth and energy storage when nutrients are available, and inhibited when fasting or in shortage of nutrients. Thus, promoting a balance between anabolism and catabolism in the cell. In contrast, mTORC2 primarily functions as an effector of the insulin/PI3K signaling pathway. mTORC2 is activated by PI3K following the activation of receptor tyrosine kinases (RTKs) or G-protein-coupled receptors (GPCRs) with growth factors, insulin or other cytokines. A cross talk between mTORC1 and mTORC2 provides regulatory check points in metabolism of energy. In relevance to lipid synthesis, mTORC1 and mTORC2 are both involved in lipid synthesis, however, through distinctive mechanisms. mTORC1 promotes de-novo lipid synthesis through the sterol responsive element binding protein (SREBP) transcription factors, which are proteolytically processed to yield mature SERBPs, that translocate to the nucleus to control the expression of metabolic genes involved in FA and cholesterol biosynthesis. mTORC2 controls FA synthesis through AKT regulation, that among other targets, promotes the activation of ATP citrate synthase (ACLY)

The present invention discloses a remarkable increase in the density of low-diameter axons within WMTs localized to the dorsal striatum of old and symptomatic α-Syn tg mouse brains. The present inventors found that α-Syn expression increases the length of main axon and collaterals through its associations with membrane PI4,5P2. Using axonal and synaptic markers, the inventors found higher glutamatergic immunoreactivity, within corticostriatal WMTs and terminals, in the caudate of postmortem human brains with early PD. The results support a role for glutamatergic plasticity early in PD. Furthermore, the invention discloses a role for α-Syn protein in regulating the activation of the PI3K/AKT and mTORC2 activity, that explains axon growth. Finally, the invention highlights over activation of PI3K/AKT pathway as a target for Parkinson's disease therapy.

### SUMMARY

The present invention, in some embodiments, is based, in part, on the finding that there are differences in PI4,5P2 levels in blood from α-Syn−/− and WT mice. This finding led to the realization that PI4,5P2 levels, and those of the related phosphoinositides, can be used, alone or together with other biomarkers, to detect various parameters related to Parkinson's in a subject.

The present invention, in some embodiments, is further based, in part, on the finding that PI4,5P2 featured higher levels in axons of α-Syn mouse brains, and higher PI, PI4P and PI4,5P2 signals were detected in α-Syn over-expressing cells compared with control cells. These findings led to the understanding that decreasing the PI4,5P2 levels, directly or via decreasing the levels of its precursor PI4P may be of therapeutic relevancy for the treatment of pathologies characterized by high levels of α-Syn.

Further, the inventors specifically showed that a higher degree of activated AKT is detected in human PD than control human brains, in A53T α-Syn tg mouse brains, and in cell lines over expressing α-Syn.

In accordance with activation of AKT, the inventors showed that higher PI3,4,5P3 (PIP3), PI3,4P2 and PI4,5P2 levels are detected in α-Syn expressing cells, and a higher degree of phosphorylated ACLY, that is down-stream of PI3K/AKT/mTORC2 pathway.

Importantly, the inventors showed that inhibiting members of PI3K class I (using a combination of genetic CRISPR-CAS) and pharmacological inhibitors), abolished the effects of α-Syn to enhance the activation of PIP3 synthesis and in complement, inhibited AKT phosphorylation. The current data provide evidence to support an involvement of protein palmitoylation in α-Syn effect to facilitate PI3K activity.

According to a first aspect, there is provided a method for determining whether a subject is afflicted with or at increased risk of developing any one of: an alpha-synuclein related disease, Parkinson's disease, and any combination thereof, the method comprising determining an amount of at least one phosphoinositide in a sample obtained or derived from a subject, wherein a decrease or an increase in the amount of the at least one phosphoinositide compared to a baseline is indicative of the subject being afflicted with or at increased risk of developing any one of the alpha-synuclein related disease, the Parkinson's disease (PD), and any combination thereof.

According to another aspect, there is provided a method for treating a subject afflicted with any one of: an alpha-synuclein related disease, PD, and any combination thereof, comprising the steps: (a) determining whether at least one phosphoinositide is present in an amount: (i) decreased below a baseline; or (ii) increased above a baseline, in a sample obtained or derived from the subject; and (b) administering to the subject determined as having decreased or increased amount of the at least one phosphoinositide below or above the baseline, respectively, a therapeutically effective amount of pharmaceutical composition comprising any one of: an agent suitable for inhibiting or reducing alpha synuclein activity, pathogenicity, or both, an agent suitable for anti PD therapy, and any combination thereof, thereby treating the subject afflicted with any one of: an alpha-synuclein related disease, PD, and any combination thereof.

According to another aspect, there is provided method for ameliorating or treating a subject afflicted with any one of: an alpha-synuclein related, PD, and any combination thereof, comprising administering to the subject a therapeutically effective amount of a pharmaceutical composition comprising an agent characterized by being capable of modifying any one of: non-steady state levels of PI4,5P2, PI4,5P2-related signaling, and both, thereby treating the subject afflicted with an alpha-synuclein related disease.

According to another aspect, there is provided a kit for diagnosing, prognosing, or both, any one of: an alpha-synuclein related disease, PD, and any combination thereof, in a subject, the kit comprising: (a) at least one phosphoinositide antagonist; and: (b) a baseline control; (c) a calibrating control; or (d) (b) and (c).

In some embodiments, determining comprises quantifying a level of decrease or increase in the amount of the at least one phosphoinositide compared to a predetermined calibrating control, wherein an increase or decrease of the level compared to the predetermined calibrating control is indicative of the severity or prognosis of the alpha-synuclein related disease, the PD, and any combination thereof, in the subject.

In some embodiments, the subject is prodromal or pre-symptomatic to the alpha-synuclein related disease.

In some embodiments, the subject is being treated for the alpha-synuclein related disease, the PD, and any combination thereof.

In some embodiments, the method is for monitoring responsiveness of the subject to anti alpha-synuclein related disease therapy, anti PD therapy, or both, wherein an increase or decrease of the level compared to the predetermined calibrating control is indicative of responsiveness of the subject to the therapy.

In some embodiments, the method further comprises a monitoring step (c) comprising at least once determining the amount of the at least one phosphoinositide in sample obtained or derived from the administered subject.

In some embodiments, the sample is selected from the group consisting of: whole blood or any fraction thereof, erythrocytes, platelets, saliva, septum, tears, and feces.

In some embodiments, the at least one phosphoinositide is selected from the group consisting of: PI4,5P2, PI, PI4P, and PI3,4,5P3.

In some embodiments, the determining comprises contacting the sample obtained or derived from the subject with a phosphoinositide antagonist having specific binding affinity to: PI3P, PI4P, PI5P, PI4,5P2, PI3,4P2, PI3,5P2, or PI3,4,5P3.

In some embodiments, the at least one phosphoinositide is PI4,5P2.

In some embodiments, the antagonist has specific binding affinity to: PI4,5P2.

In some embodiments, the antagonist comprises any one of: an antibody, a phosphoinositide binding protein or a binding domain thereof, a soluble receptor, and any functional fragment thereof.

In some embodiments, the modifying comprises increasing or decreasing any one of: the non-steady state levels of PI4,5P2 to physiological steady state levels, the PI4,5P2-related signaling, and both, in the subject.

In some embodiments, the method further comprises a monitoring step proceeding the administering, comprising at least once determining the amount of at least one phosphoinositide in a sample obtain or derived from the administered subject.

In some embodiments, the determining comprises contacting the sample obtained or derived from the subject with a phosphoinositide antagonist having specific binding affinity to PI4,5P2.

In some embodiments, the antagonist comprises any one of: an antibody, a phosphoinositide binding protein or a binding domain thereof, a soluble receptor, and any functional fragment thereof.

In some embodiments, the antagonist comprises any one of: an antibody, a phosphoinositide binding protein or a binding domain thereof, a soluble receptor, and any functional fragment thereof.

In some embodiments, the alpha-synuclein related disease is selected from the group consisting of: PD, Lewy body dementia, Alzheimer's disease (AD), multiple system atrophy, NIEMANN-PICK-type A, and any combination thereof.

In some embodiments, the PD comprises PD with dementia (PDD).

In some embodiments, the agent is 3-(4-carbamoylphenyl)-N-(5-cyanopyridin-2-yl)-N-methylpyrazolo[1,5-a]pyridine-5-carboxamide, 5-2-amino-1-(4-morpholinophenyl)-1H-benzo[d]imidazol-6-yl)-N-(2-fluorophenyl)-2-methoxypyridine-3-sulfonamide, or a combination thereof.

In some embodiments, the kit further comprises instructions for determining an amount of the at least one phosphoinositide, in a sample obtained or derived from a subject.

In some embodiments, the kit further comprises any one of: an agent characterized by being capable of inhibiting or reducing alpha synuclein activity, pathogenicity, or both, an anti-PD therapeutic agent, and both, in a subject in need thereof.

In some embodiments, the kit is for ameliorating or treating a subject diagnosed or prognosed for the alpha-synuclein related disease, the PD, and any combination thereof.

In some embodiments, the kit is for monitoring any one of: disease progression or regression, responsiveness of a subject to therapy, or both, of any one of: the alpha-synuclein related disease, the PD, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1J include micrographs and graphs showing ultrastructure of cross-sectioned axons in white matter tracts (WMTs). (1A) Lower magnification of a coronal mouse brain section, stained with Luxol Fast blue (Sigma-Aldrich), showing the striatum. CC, corpus callosum; LV, lateral ventricle; WMTs shown as dark spots over the lightly stained striatal grey matter. Bar=1 mm. (1B) Cross section of a WMT in the dorsal striatum, consisting of myelinated axons. Image obtained by transmission electron microscope (TEM). Bar=4 μm. (1C) TEM images of coronal brain sections containing WMTs from A53T α-Syn and control mouse brains, at 4 months (healthy mice) or 12 months of age (symptomatic). Bar=2 μm. (1D) bar graph showing the diameter (in μm) of cross-sectioned axons in WMTs and (1E) The number of axons per μm² area of WMTs (axon density). Mean±SE n=4 brains, 5-8 WMTs per brain. (1F) Intact myelin ultrastructure of a cross-sectioned axon in a WMT of 12 months old A53T α-Syn tg mouse brain. Bar=200 nm. (1G) A semi-thick A53T α-Syn mouse brain section (1 μm), stained with methylene blue (Sigma-Aldrich), showing cross-sectioned axons in a WMT. Bar=20 μm. (1H) Graph showing axon density in WMTs of Thy-1 hWT α-Syn and control mouse brains at 10-12 months of age. Determined in semi-thick sections (1 μm) stained with methylene blue. Mean±SE of n=4 brains, 8-10 WMTs per brain. (1I) and (1J) TEM images showing sagittal brain sections across the axons in corpus callosum of 12-14 months old A53T and age-matched control (C57BL/6JOlaHsd) mouse brains.

FIGS. 2A-2G include fluorescent micrographs and graphs showing elongated axons and collaterals in primary cortical neurons expressing α-Syn associate with higher levels of phosphatidylinositol 4,5-bisphosphate (PI4,5P2). (2A) Primary cortical cultures from α-Syn^{-/-} (C57BL/6JOlaHsd) mouse brains, virally transduced either with WT α-Syn, A53T α-Syn, K10,12E α-Syn, or a mock-GFP vector. Cells were fixed at 4 DIV and immunoreacted with antibodies against α-Syn (MJFR1, green), α-Tubulin (red) and PI4,5P2 (white). Direct fluorescence was captured for GFP (green). Bar=25 μm. (2B) Graph showing the average axon length (in μm); (2C) Total length of collaterals per axon (in μm); and (2D) PI4,5P2 levels within the axon and its collaterals (per μm² area) quantified by Fiji (Image J) program. Mean±SE; n>22 cells; *, P<0.05. (2E) A primary cortical neuron as in (2A), transduced with a mock-GFP (green); immunoreacted with anti-GAP-43 (blue) and stained with rhodamine-phalloidin (red). Bar=10 μm. (2F) A neuron as in (2E) expressing WT α-Syn and immunoreacted with anti α-Syn (MJFR1, green) and anti α-tubulin (blue) abs and stained with rhodamine-phalloidin (red). Shown an entire cell and a zoom on axon collaterals. Bar=5 μm. No differences in number of growth cones per axon (in μm) were detected. (2G) Graph bar showing quantitation of growth cones per μm axon in WT α-Syn and GFP expressing neurons. Mean±SD of n=12-15 cells.

FIGS. 3A-311 include graphs, micrographs, and fluorescent micrographs, showing that α-Syn expression regulates PI4,5P2 levels. (3A) SK-mel2 cells transduced with viral particles expressing either sh-SNCA or a control (sh-Cntrl). 3-7 days post transduction, samples of lysed cells (80 μg protein) were analyzed by Western blotting for α-Syn expression using MJFR1 antibody. (3B) SK-mel2 cells transduced as in (3A) and analyzed by FACS for α-Syn, using anti α-Syn antibody MJFR1; and (3C) using anti PI4,5P2 antibody. (3D) HEK 293T cells were transfected either with WT α-Syn, A53T or K10,12E α-Syn mutations, or mock transfected. 48 hours post transfection, PI4,5P2 levels were determined by FACS, using anti PI4,5P2 antibody. Cells were gated based on positive signal for α-Syn detected by MJFR1 antibody. Mean±SE, n>3,000 cells; *, P<0.05, one way ANOVA. (3E) HeLa cells, co-transfected with WT α-Syn and PH-PLCδ1-GFP. Control cells transfected with the mock plasmid together with PH-PLCδ1-GFP. Cells were incubated with Alexa-647 Concanavalin (Con)A for visualization of plasma membrane (PM) and processed for ICC with anti α-Syn antibody, C20. Showing the co-localization of PH-PLCδ1-GFP and 647-ConA signals (direct fluorescence). Bar=10 μm. (3F) Calculated plasma membrane to cytosol ratio of the PH-PLCδ1-GFP signal in HeLa cells, co-expressing the indicated constructs. Mean±SE of n>15 cells. *, p<0.05, one way ANOVA. (3G) High magnification of paraffin embedded mouse brain section containing the hippocampus, analyzed by IHC and immunoreacted with anti PI4,5P2 ab (green). Section stained with DAPI (blue). Nomarski image (gray) shown to demonstrate PI4,5P2 signal on PM. Bar=10 μM. (3H) Primary cortical neurons from WT C57BL/6 or α-Syn −/− (C57BL/6JOlaHsd) at 14 DIV. Cultured neurons analyzed by ICC and immunoreacted with anti NF-200 and anti PI4,5P2 abs. Bar graph shows signal ratio of PI4,5P2 to NF200. Mean±SD, n=5 fields, consisting of >10 neuronal cell bodies each; *, P<0.05, t-test.

FIGS. 4A-4J include micrographs and graphs showing that α-Syn effect to elongate axons and collaterals is mediated through PI4,5P2. (4A) PI4,5P2 levels determined by FACS, in HEK 293T cells, following 48 hours from cell transfection with α-Syn and/or Sj-1 phosphatase, as indicated. Mean±SD of two independent experiments, n>3,000 cells in each treatment; P<0.05, one-way ANOVA. (4B) PI4,5P2 levels determined by FACS in HEK 293T cells transfected with α-Syn and/or PIPKIγ as indicated. Mean±SD of two independent experiments, n>3,000 cells in each treatment; P<0.05, one-way ANOVA. (4C) Primary cortical cultures from α-Syn−/− mouse brains, electroporated at day of preparation to co-express WT α-Syn together either with PIPKIγ-GFP, Sj-1-GFP or mock-GFP expressing vectors (as indicated). Cultures were fixed and immunoreacted with anti α-Syn MJFR1 ab and α-tubulin. GFP signal was captured by direct fluorescence. Tubulin signal is shown. Bar=25 μm. (4D) The average length of axons (in μm). (4E) Total length of collaterals per axon (in μm); and (4F) PI4,5P2 levels within the axon and its collaterals (per axon area) quantified by Fiji program. Mean±SE; n>25 cells; *, P<0.05 one-way ANOVA. (4G) HEK293T cells transfected to express mCherry NIR2 or mock transfected (cntrl). Western blot showing mCherry Nir2 signal detected with anti Nir2 ab (Abcam). PI4,5P2 levels determined by FACS. Mean±SE, n>3,000 cells; *, P<0.05, t-test. (4H) Tet-on inducible SH-SYSY cells for inducible α-Syn expression were infected with viral vectors expressing either shNIR2 or shCntrl (Mission, Sigma-Aldrich). α-Syn expression induced with doxycycline (1 μM/ml) or non-induced. PI4,5P2 levels determined by FACS. Mean±SD of n=3 different experiments, >3,000 cells in each treatment. (4I) Quantitative PCR (qPCR) detection of α-Syn following its induced expression in SH-SYSY cells without and with the addition of doxycycline for 72 hours. α-Syn mRNA levels normalized to the levels of G6PD gene detected in the same sample. (4J) Quantitative detection of NIR2 expression by qPCR in these cells.

FIGS. 5A-5E include fluorescent micrographs, micrographs, and graphs showing differences in WMTs between α-Syn transgenic and control mouse brains. (5A) Paraffin embedded coronal brain section (6 μm) from A53T α-Syn tg mouse, immunoreacted with anti NF-200 (green) and anti PI4,5P₂ (red) antibodies. Showing a WMT and the localization of the immunoreactivity to axon membrane. (5B) Graph showing the immunoreactive signal ratio obtained for PI4,5P2 and NF-200 within WMTs (per area) for A53T α-Syn or Thy-1 hWT α-Syn tg mouse models, presented as percent of age- and genotype-matched control mice set at 100% (represented by a vertical line). Mean±SE, n=4 brains, *, p<0.05, one-way ANOVA. (5C) Paraffin embedded coronal brain sections (6 μm) of A53T α-Syn and control (α-Syn^{-/-}, C57BL/6JOlaHsd) mouse brains at 12 months of age, containing the dorsal striatum, immunoreacted either with antibodies against axonal markers SMI-32, SMI-31 and NF200 or anti APP antibody, a marker for axonal damage. Bar=50 μm. (5D) Quantification of the immunoreactivity obtained in (C) in WMTs of A53T α-Syn tg and control (α-Syn^{-/-}, C57BL/6JOlaHsd) mouse brains, at 2, 8 and 12-14 months of age. Vertical line represents age-matched control mice, set at 100% for each of the tested antibodies. Mean±SE; n=5 mouse brain. *, P<0.05, one-way ANOVA. (5E) Consecutive brain sections (as in 5C and 5D) co-immunoreacted with syn303 anti α-Syn and anti SMI-32 antibodies. Bar=20 μM.

FIGS. 6A-6G include fluorescent micrographs, micrographs, and graphs showing axonal plasticity in corticostriatal connections at early stages of PD. (6A) SMI-32, vGluT1 and TH immunoreactive signals localized to the caudate of early PD (unified stage IIa and IIb; and control brains. N=5 each). (6B) graph (as in 6A) showing advanced PD (Braak stages 5-6; n=5) and control brains (n=7). Mean±SD of 5-7 fields. *, P<0.05, t-test. (6C) IHC of caudal WMTs in a control (Male, 89 years) and an advanced PD brain (Male, 82 years, Braak stage 6), immunoreacted with anti SMI-32 antibody. (6D) Positive correlation (Pearson's r value=0.83) between the immunoreactive signals obtained for vGluT1 and SMI-32 for PD cases. (6E) A caudal WMT probed for α-Syn pathology by IHC. A control brain (male, 84 years) and an advanced PD (male, 75 years, Braak stage 5) brain. (6F) IHC of caudal WMTs in a control brain (male, 84 years) an early PD brain (male, 72 years, unified stage IIb) and an advanced PD brain (Male 75 years, Braak stage 5). Brain sections co-immunoreacted with anti-filament-α-Syn and SMI-32 antibodies. Bar=20 μm. (6G) Filament-α-Syn and SMI-32 signals as in (6F). Mean±SD of n=10-15 WMTs.

FIGS. 7A-7I include fluorescent micrographs, micrographs, and graphs showing the dopaminergic system in A53T α-Syn tg mouse brains. (7A) Coronal brain sections (6 μm², bregma +1.3-(−3.8)) of A53T α-Syn tg mouse (14 months), immunoreacted with anti-Tyrosine hydroxylase (TH) antibody. CC, corpus callosum; CPu, caudate putamen; LV, lateral ventricle; mfb, medial forebrain bundle; NAc, nucleus accumbens; OT, olfactory tubercle; VP, ventral pallidum; VTA, ventral tegmental area. 3V, third ventricle. Bar=500 μm. (7B-7C) Higher magnification of brain sections (as in 7A). (7B) SNc, Bar=25 μm. (7C) Striatal WMT. Bar=20 μm. (7D) Protein samples (30 μg) of striatal homogenates from 12-14 months old A53T α-Syn tg and age matched α-Syn^{-/-} control mouse brains analyzed by western blotting. Blot immunoreacted with antibodies for dopamine transporter (DAT); Tyrosine hydroxylase (TH); β-actin; and synaptophysin (SP). A representative blot out of two. N=5 brains in each genotype. (7E) Graph showing quantitation of blots in (7D) Values to normalized to β-actin levels in the same sample. Vertical bar represents control brains, set at 100%. (7F) Quantitative value of western blot as in (7D) reacted with anti vGluT1 ab. (7G) immunohistochemistry (IHC) of paraffin embedded mouse brain section co-immunoreacted with anti vGlut1 (red) and anti NF-200 (green) abs. Bar=25 μm. (7H) IHC of a paraffin embedded section containing the olfactory tubercle from an A53T α-Syn tg mouse brain at 12 month of age, co-immunoreacted with anti TH ab. (7I) Quantification of immunoreactivity obtained by IHC in the olfactory tubercle. Paraffin brain sections of A53T α-Syn tg and control 12-14 months old mice, immunoreacted with anti TH antibody as in (7H). Mean±SD of n=4 mouse brain. *, P<0.05, t-test. Bar=100 μm.

FIG. 8. includes a scheme showing a non-limiting outline showing that α-Syn enriches axon membrane with the acidic phosphoinositide PI4,5P2 to facilitate axon outgrowth and arborization. In brains affected with PD, over expression or mutations in α-Syn increase axon density at early stages of the disease and enhance glutamatergic plasticity in caudal white matter tract (WMT). However, with disease progression, accumulation of damage is leading to degeneration and loss of axons within WMT.

FIGS. 9A-9H include fluorescent micrographs and graphs showing that α-Syn expression enhances axonal growth in primary hippocampal, mesencephalic, and cortical neurons. (9A) Primary hippocampal cultures from α-Syn^{-/-} (C57BL/6JOlaHsd) mouse brains, virally transduced either with WT α-Syn, A53T α-Syn, K10,12E α-Syn, or a mock-GFP vector. Cells were fixed at 4 DIV and immunoreacted with antibodies against α-Syn (MJFR1, green), α-Tubulin (white) and PI4,5P2 (red). Direct fluorescence was captured for GFP (green). Bar=25 μm. (9B) Graph showing the average length of the main extension designated as axons (in μm); (9C) Total length of collaterals per axon (in μm); and (9D) PI4,5P2 levels within the main axon and its collaterals (per 1 μm² area) quantified by Fiji (Image J) program. Mean±SE; n>17 cells; *, P<0.05; Bar=5 μm. (9E) Cortical neurons cultured and treated as in FIG. 2, expressing A30P α-Syn mutation, WT α-Syn or GFP (mock). Mean±SE; n>15 cells; *, P<0.05. (9F-9G) Primary mesencephalic neurons at 4 DIV from α-Syn^{-/-} mouse brains. Neurons were virally transduced to co-express α-Syn and a mock plasmid, α-Syn and PIPKIγ or α-Syn and Sj-1. Control neurons expressed a mock GFP plasmid, Cultured neurons immunoreacted (as in 9A) with anti TH antibody to verify dopaminergic properties, indicated˜90% positivity. (9F) Average length of the main extension designated as axons (in μm); (9G) Total length of collaterals per axon (in μm); and (9H) PI4,5P2 levels within the main axon and its collaterals (per 1 μm² area) quantified by Fiji (Image J) program. Mean±SE; n>20 cells; *, P<0.05.

FIGS. 10A-10C include fluorescent micrographs showing the specificity of PI4,5P₂ signal. (10A) Specificity of PI4,5P₂ signal is detected by ICC in primary neurons, following activation of muscarinic receptors with carbachol (Sigma Aldrich) and PLC-mediated hydrolysis of PI4,5P₂. Primary cortical neurons at 5 DIV, treated with 1 mM carbachol for 12 minutes. Bar=20 μM. (10B) Higher magnification of a cortical neuron (as in 10A), demonstrating PI4,5P₂ signal on PM surrounding cell body. (10C) HEK 293T cells transiently expressing the rapamycin-induced translocatable CFP-PIPK kinase. CFP-PIPK fluorescence is either cytosolic or on the PM in DMSO or rapamycin (respectively) treated cells. CFP-PIPK (blue) is directly detected at 485 nm and PI4,5P₂ detected by ICC with anti PI4,5P₂ ab (red). Bar=10 μM.

FIG. 11 includes micrographs showing vGluT1 and TH immunoreactivity in control and PD brains. Slides containing caudate of a control (Male 85 years, 3 h PMI) and an early PD case (male 83 years, 2 h PMI, Unified stage IIa), immunoreacted with anti vGluT1 (red) and anti TH (green) antibodies. Bar=10 μm.

FIGS. 12A-12H include fluorescent micrographs and graphs showing that α-Syn colocalizes with phosphorylated AP2 and PIP2 on clathrin-coated pits. (12A) SK-Mel2 cells were processed for the detection of the immunoreactive signals of endogenous α-Syn (ab 21976 ab, red), phosphorylated Thr156 μ2 subunit of AP2 (pAP2; gray) and PI4,5P₂ (green) by immunocytochemistry (ICC). Bar=10 μm. (12B) SK-Mel2 cells treated with LP-935509 (10 μM, for 3 hours), to inhibit the phosphorylation of the μ2 subunit of AP2 and processed for ICC as in (12A). Bar=10 μM. (12C) SK-Mel2 cells infected with lentivirus encoding shSNCA to silence α-Syn expression. Cells were treated with LP-935509 inhibitor as in (12B) and processed for ICC. Bar=10 μM. (12D) SK-Mel2 cells as in (12A), however, immunoreacted for PI3,4P₂ detection (green). Bar=5 μM. (12E) Higher magnification of the image shown in (12D), focusing on plasma membrane. Arrows indicate spots of colocalization for α-Syn/pAP2/PI3,4P₂. (12F) HEK 293T cells, transfected to express the specified PIP-metabolizing proteins. Cells analyzed by FACS to immunodetect PI4,5P₂ levels. N>2,000 cells in each group; mean±SE * P<0.01, t-test. (12G) Cells analyzed by FACS as in (12F) to detect PI3,4P₂ signal. N>2,000 cells; mean±SE * P<0.01, t-test. (12H) SK-Mel2 cells processed for ICC as in (12A) and co-immunoreacted with anti α-Syn (MJFR1) and anti-PIP abs (Echelon). Colocalization of the signal obtained for α-Syn with the specified PIP was quantified and normalized to total α-Syn positive spots. Colocalization is reduced in controls cells that express the specific PIP-phosphatase. N>22 cells; mean±SE.

FIGS. 13A-13C include fluorescent micrographs showing α-Syn and PIP2 at CCP in mouse brains. (13A) Paraffin embedded section (6 μm) containing the hippocampus of a 2-months old A53T α-Syn tg mouse, immunoreacted with anti α-Syn ab (ab21976, red), phosphorylated Thr156 μ2 subunit of AP2 (pAP2; blue) and PI4,5P₂ (green). Bar=20 μm. (13B) Magnification of the image in (13A) to demonstrate the colocalization between α-Syn (red), pAP2 (blue) and PI4,5P₂ (green) surrounding cell nucleus. Nuclei are demonstrated by the dotted ovals. Bar=10 μM. (13C) Paraffin embedded section (6 μm) of 2 months old A53T α-Syn tg mouse (as in 13A), immunoreacted with anti α-Syn (ab21976, red), phosphorylated Thr156 μ2 subunit of AP2 (pAP2; blue) and PI3,4P₂ (green). Bar=20 μm.

FIGS. 14A-14G include fluorescent micrographs and graphs showing α-Syn increases PIP2 levels and endocytosis of 568-Tf. (14A) The kinetics of Alexa-568-Transferrin (568-Tf; 25 μg/ml) endocytosis in α-Syn or mock transfected HEK293T cells. Cells were incubated in parallel with 568-Tf for 0-12 minutes at 37° C. Graph presenting the sum of signal in endocytic vesicles (above threshold). N>30 cell; mean±SE of α-Syn (circles) and mock (squares) expressing cells. *, P<0.05; t-test with Bonferroni correction for multiple comparisons. (14B) SK-Mel2 cells infected with lentivirus encoding shSNCA or shCntrl. 568-Tf was applied to cells for 7 minutes and cells were processed for the detection of 568-Tf (red) endocytosis as in (14A). α-Syn detected with an anti-α-Syn ab (Syn211; green). Bar=10 μm. (14C) SK-Mel2 cells expressing shSNCA or shCntrl (as in (14A)). The immunoreactive signals for α-Syn, PI4,5P₂, PI3,4P₂ detected by ICC; 568-Tf was detected as in (14B). Mean±SE of n=17-33 cells per treatment;*, P<0.05 t-test, with Bonferroni correction for multiple comparisons. (14D) Cells expressing PH-PLCδ1-GFP (green, direct fluorescence) and immunoreacted with anti α-Syn ab (Syn211; red). Bars=10 μm. (14E) The PH-PLCδ1-GFP signal ratio (plasma membrane to cytosol) determined in SK-Mel2 cells expressing shCntrl or shSNCA (n>15 cells per treatment, mean±SE; P<0.05 t-test). (14F) Inducible, Tet-on SH-SY5Y α-Syn-expressing cells incubated with doxycycline (1 μg/ml) for 72 h or with the DMSO solvent. Cells processed for ICC and immunoreacted with anti α-Syn ab (MJFR1; Red). DAPI staining depicts nuclei (blue). Bars=20 μm. (14G) α-Syn expression was induced with doxycycline (1 μg/ml) for 72 h in the inducible SH-SY5Y cells. Control cells were treated in parallel with the solvent. Cells immunoreacted with anti-PIP ab (Echelon) as specified and analyzed by FACS. Results presented as percent of control cells, with control mock-vector, set at 100%. N>2,000 cells per treatment; mean±SE; * P<0.05; t-test with Bonferroni correction for multiple comparisons.

FIGS. 15A-15E include fluorescent micrographs, a micrograph, and graphs showing that the enhancing effect of α-Syn on CME is PI4,5P₂ dependent. (15A) HEK 293T cells, transfected to co-express the three plasmids: Lyn-FRB, FKBP-CFP-Inp54p and WT α-Syn. On day of experiment, cells were serum starved for 90 minutes and then incubated for 7 minutes with 568-Tf and rapamycin (500 nM) to induce the recruitment of the Inp45p phosphatase (P-ase) to the plasma membrane along with internalization of 568-Tf. Control cells were treated in parallel with DMSO (0.5% v/v). Cells were then acid-washed, fixed and immunoreacted with anti α-Syn ab (MJFR1; Green). Direct fluorescence for CFP (P-ase; Grey) and 568-Tf (Red). (15B) HEK 293T cells expressing Lyn-FRB and FKBP-CFP-Inp54p were treated either with rapamycin or DMSO as described in (15A), followed by immunoreaction with anti PI4,5P₂ ab (Echelon). A quantification of PI4,5P₂ signal is shown (n>9 cells per treatment; mean±SE; * P<0.01 t-test). (15C) Graph showing quantification of internalized 568-Tf signal in cells transfected and treated as in (15A). Cells transfected with a mock vector (white bars, squares) or α-Syn (black bars, circles). Mean±SE of n=4 experiments; n>18 cells per treatment in each experiment; * P<0.05 t-test; with Bonferroni correction for multiple comparisons. (15D) The inducible Tet-on SH-SY5Y were infected with shNir2 or a control shRNA (shCntrl). Protein samples were analyzed by a western blot, immunoreacted with anti-Nir2 ab (Abcam). (15E) Cells as in (15D) were induced to express α-Syn with doxycycline (Dox, 1 μg/ml, for 72 h) or treated in parallel without Dox. Cells were then processed for ICC to measure 568-Tf endocytosis, as described in (15C). Mean±SE, 3 experiments, n=20-25 cells per treatment; * P<0.05, t-test. Sister cultures were immunoreacted with anti PI4,5P₂ ab and analyzed by FACS. Mean±SE of 3 experiments, each consisting of n>2,000 cells per sample; * P<0.05 t-test; with Bonferroni correction for multiple comparisons.

FIGS. 16A-16B include graphs showing that α-Syn mutations correlate endocytosis of transferrin with changes in plasma membrane levels of PI4,5P₂. (16A) HEK 293T cells were transfected to express the indicated α-Syn forms. Cells were processed for 568-Tf endocytosis by ICC (n>15 cells per treatment). Sister cultures were immunoreacted with anti-PI4,5P₂ ab or anti-PI3,4P₂ ab and analyzed by FACS. Plasma membrane PI4,5P₂ determined by calculating the membrane to cytosolic signal ratio of PH-PLCδ1-GFP in cells expressing WT α-Syn or the specified α-Syn mutations. Vertical line represents WT α-Syn, set at 100%. Mean±SE of n=3-4 experiments. In each experiment n=20-25 cells (ICC) or >2,000 cells (FACS) per treatment. *, P<0.05, t-test with Bonferroni correction. CORREL, correlation coefficient. (16B) Cells as in (16A) showing α-Syn signal determined by FACS, presented as percent of the signal determined for WT α-Syn in each experiment. Mean±SD, n=3-4 experiments, >2,000 cells for each α-Syn construct.

FIGS. 17A-17G include graphs showing that α-Syn mutations differentially affects SV endocytosis and neuronal PI4,5P₂ levels. (17A) Hippocampal neurons at 13 DIV, prepared from α-Syn^{-/-} mouse brains were infected to express sypHy and mCherry, and either WT α-Syn or a mock plasmid. Neurons were stimulated for 15 seconds at 20 Hz (300 stimuli) at room temperature and the change in sypHy fluorescence in the synaptic puncta was recorded. The baseline fluorescence prior to stimulation (F0) was subtracted. Fluorescence was normalized to the total pool of vesicles (Fₘₐₓ) measured at the end of the measurements by exposure to NH₄Cl-saline. Mean±SE of n=6 slides per treatment (50 synapses per image, in 3 independent experiments). (17B) As in (17A), but the bath included 10 μM BafA, and 2,400 stimuli were applied at 20 Hz. (17C) Shown is the fluorescence measured 120 seconds after starting stimulation (as in (17B)), normalized by the total pool of vesicles (Fₘₐₓ), mean±SE; n=12-20 slides, 30-50 synapses per image). P<0.001 t-test. (17D) Cells as in (17A). Shown is AF scaled to the peak fluorescence (Fₚₑₐₖ). Mean±SE; n=6 slides per treatment (50 synapses per image, in 3 independent experiments). (17E) Fractional peak release values (Fₚₑₐₖ/Fₘₐₓ) for each of the specified α-Syn form. Shown are mean±SE values; n=3-6 slides per treatment (30-50 synapses per image, 3 experiments). * P<0.05 t-test; with Bonferroni correction for multiple comparisons. (17F) Graph showing the calculated decay constant resembling the rate of endocytosis with each of the specified α-Syn form or the control plasmid. N=3-6 slides per treatment (30-50 synapses per image, 3 experiments). *P<0.05 t-test; with Bonferroni correction for multiple comparisons. (17G) Hippocampal neurons expressing the indicated α-Syn forms or a mock plasmid were processed for ICC at 13 DIV and immunoreacted with anti α-Syn ab (MJFR1) and anti PI4,5P₂ ab (Echelon). Graph showing the quantification of PI4,5P₂ in α-Syn positive neurites. Mean±SE; n=7-16 fields per treatment * P<0.05 t-test; with Bonferroni correction for multiple comparisons.

FIG. 18 includes a graph showing CME in primary neurons. Primary neurons from WT mice (C57BL/6J) or α-Syn^{-/-} (C57BL/6JOlaHsd) mice were transfected to express the indicated α-Syn forms or a mock GFP plasmid. At 8 DIV cells were starved for 90 min, incubated for 7 min with 568-Tf and then acid-wash. Cells were immunoreacted with anti α-Syn ab (Syn211). The signal obtained for Tf-568 in neuronal somas was quantified. N=17-36 cell somas per treatment * P<0.05 t-test; with Bonferroni correction for multiple comparisons.

FIGS. 19A-19D include fluorescent micrographs showing anti α-Syn antibodies reacted with endogenous α-Syn in SK-Mel2 cells. Cells were incubated with the following anti α-Syn antibodies: MJFR1 (1:2,000; 19A), Syn211 (1:750; 19B), LB509 (1:250; 19C) and ab21976 (1:330; 19D).

FIGS. 20A-20B include graphs showing levels of phosphatidylinositol(s) in blood cells extracts. (20A-20B) PI4,5P₂ detected in blood cells extracts. PI4,5P₂ levels in blood cells from control (CON) and PD patients were determined by enzyme-linked immunosorbent assay (ELISA). Cells were extracted according to extraction protocol 1 (20A) or extraction protocol 2 (20B). The detected levels were normalized to total phosphates in the sample, as determined by Malachite green.

FIGS. 21A-21B include micrographs and a vertical bar graph showing AKT activation in human PD brains. A higher degree of phosphorylated AKT at Thr 308 and Ser 473 out of total AKT is detected in postmortem PD brains than in control human brains (age- and gender-matched, without neurodegeneration). Frozen tissue was obtained from the Multiple Sclerosis and Parkinson's Tissue Bank, tissue containing the caudate (striatum). Post mortem intervals are 3-12 hours. Tissue was homogenized and analyzed by Western blotting (21A). The immunoblot was reacted with the indicated antibodies (Cell Signaling. Ornat, Rehovot, Israel). A representative image. Quantification of 21A is provided in 21B. Mean±SE of n=12 brains in each group, analyzed in three different dates, *, p<0.05, one way ANOVA.

FIGS. 22A-22C include micrographs and a multiple vertical bar graphs showing AKT and ACLY phosphorylation in mouse brains tg for α-Syn. A higher degree of phosphorylated AKT at Thr 308 or Ser 473 out of total AKT (22A); and P-ACLY (Ser 454; 22B) out of total ACLY [down-stream of mTORC2] is detected in A53T α-Syn tg than in control mouse brains. Mice are 3 months old, showing whole brain extracts of two mice for each genotype. A representative image out of total of 4 mice in each genotype tested. Note that there is no difference in AKT phosphorylation between WT and α-Syn^{-/-} mouse brains, which affected the choice of cell systems in the research proposal. Quantification of 22A and 22B are provided in 22C. Mean±SD, * P<0.05, ANOVA.

FIG. 23 includes a vertical bar graph showing that α-Syn expression is associated with PI3K/AKT/mTORC2 activation in HepG2 cells. Higher levels of phosphorylated AKT (Thr308 and Ser473) and a higher degree of phosphorylated ACLY detected in HepG2 cells, infected to express α-Syn, than in control cells infected with an empty vector (EV). Cells are grown in EMEM and 8% FCS to avoid non-specific expression of α-Syn. Mean±SD of n=3 independent experiments, * P<0.05, t-test.

FIG. 24 includes a vertical bar graph showing that α-Syn increases PIP levels in serum-fed HepG2 cells. Cells were infected to express α-Syn or a mock vector (Con). Four days post-infection, cells were fixed and permeabilized before incubation with 1° and 2° abs. Cells analyzed using BD LSR Fortes Cell Analyzer, equipped with 5 lasers and the FLOWJO, LLC software. Gating is based on size and granularity, and immunoreactivity. PI3,4,5P₃, PI3,4P₂ or PI4,5P₂ detected using specific abs (Echelon; >4,000 gated cells). Mean±SD of n=3 independent experiments, *, P<0.05, one way ANOVA.

FIGS. 25A-25D include vertical bar graphs showing that silencing either PI3KCA or PI3KCB interfere with α-Syn-mediated AKT activation. (25A) HepG2 cells were co-infected with an empty vector (EV) or α-Syn expressing vector together with a CRISPR/CAS vector to silence either PI3KCA (CA, p110α) or PI3KCB (CB, p110β), or a control vector (CT). 72 hours post infection, cells were processed for the detection of PIPs by FACS or analyzed by Western blotting for the detection of AKT. Left panel shows the ratio of PI3,4,5P₃ (PIP3) to PI4,5P₂ (PIP2). Right panel shows phospho Serine 473 AKT to total AKT. (25B) The effect of PIK75 (100 nM, PI3KCA inhibitor) or TGX 221 (100 nM, PIKCB inhibitor) on PIP3/PIP2 ratio (left) or PSer473/total AKT ratio (right) as a function of α-Syn expression. Inhibitors were added for 2 hours before cells were lysed. (25C-25D) The combined effects of CRISPR/CAS and pharmacological inhibitors for PI3KCA (25C) or PI3KCB (25D) demonstrate inhibition of α-Syn-mediated increases in PI3K/AKT pathway activation. Results are mean±SD of n=3-4 trials.

FIG. 26 include micrographs showing western blot analysis of cells infected to express α-Syn or the empty vector together either with CRISPR/CAS for PI3KCA or PI3KCB as in (25A) and reacted with the indicated antibodies.

FIG. 27 includes a vertical bar graph showing that inhibitors for APT1/APT2 depalmitoylases interfere with the effects of α-Syn to enhance PIP3 levels. ML348 and ML349, inhibit the depalmitoylating enzymes Acyl protein thioesterase 1 and 2 (APT-1, APT-2, respectively). HepG2 cells were infected to express a mock vector (EV) or α-Syn. 5 days post infection, cells were serum starved for 4 hours in the presence of the indicated inhibitors: 2-Bromo-Palmitate (2BP 50 μM, an inhibitor of palmitoylation), ML348 and ML349 (10 μM each). PIP levels determined by FACS as in FIG. 4. The ratio of PI3,4,5P₃ (PIP3) to PI4,5P₂ (PIP2) is presented. A representative experiment out of n=2 highly similar experiments. Mean±SE of the replicates within one experiment, n>2,000 gated cells.

### DETAILED DESCRIPTION

### Methods of Diagnosis and Treatment

According to some embodiments, there is provided a method for determining whether a subject is afflicted with or at increased risk of developing an alpha-synuclein related disease, Parkinson's disease (PD), or both.

According to some embodiments, there is provided a method for treating a subject afflicted with an alpha-synuclein related disease, PD, or both.

In some embodiments, the method comprises determining an amount of at least one phosphoinositide in a sample obtained or derived from a subject.

In some embodiments, a decrease in the amount of the at least one phosphoinositide compared to a baseline is indicative of the subject being afflicted with or at increased risk of developing an alpha-synuclein related disease.

As used herein, the term “baseline” refers to the level of at least one phosphoinositide in a healthy subject, or any sample derived or obtained therefrom. In some embodiments, “baseline” refers to the level of at least one phosphoinositide in a subject not afflicted with or not having increased risk of developing an alpha-synuclein related disease, PD, or both.

In some embodiments, “baseline” refers to the level of at least one phosphoinositide in the subject before developing an alpha-synuclein related disease, PD, or both. In some embodiments, baseline” refers to the standard level of at least one phosphoinositide in a healthy subject.

As used herein, the term “standard” encompasses an average level of the at least one phosphoinositide as determined in a human subject.

As used herein, the terms “baseline”, “standard level”, and “steady-state level” (such as “physiological steady-state level”) are interchangeable.

A none-limiting example of a method for determining PI4,5P2 in a blood sample comprises ELISA, such as exemplified herein.

The baseline or standard amount or level of phosphoinositides in a human subject would be apparent to one of ordinary skill in the art, including methods for determining same. A non-limiting example for such a method includes, but is not limited to an enzyme-linked immunosorbent assay, such as exemplified herein.

In some embodiments, no decrease in the amount of the at least one phosphoinositide compared to a baseline is indicative of the subject not being afflicted with or at increased risk of developing an alpha-synuclein related disease, PD, or both.

In some embodiments, an increase in the amount of the at least one phosphoinositide compared to a baseline is indicative of the subject being afflicted with or at increased risk of developing an alpha-synuclein related disease, PD, or both.

In some embodiments, no increase in the amount of the at least one phosphoinositide compared to a baseline is indicative of the subject not being afflicted with or at increased risk of developing an alpha-synuclein related disease, PD, or both.

In some embodiments, determining comprises quantifying a level of decrease or increase in the amount of the at least one phosphoinositide compared to a predetermined calibrating control. In some embodiments, quantifying comprises determining the amount and comparison thereof to a predetermined calibration scale, curve, score, or any equivalent thereof.

In some embodiments, a decrease or an increase of at least 2%, 3%, 5%, 7%, 8%, 9%, 10%, 12%, 14%, 15%, 17%, 20%, or any value and range therebetween, compared to a baseline, is indicative of the subject being afflicted with or at increased risk of developing an alpha-synuclein related disease, PD, or both. Each possibility represents a separate embodiment of the invention. In some embodiments, a decrease or an increase of 1-3%, 1-5%, 2-7%, 2-8%, 2-9%, 1-10%, 2-12%, 2-14%, 1-15%, 2-17%, or 1-20%, compared to a baseline, is indicative of the subject being afflicted with or at increased risk of developing an alpha-synuclein related disease, PD, or both. Each possibility represents a separate embodiment of the invention.

In some embodiments, an increase or a decrease of the level compared to a predetermined calibrating control is indicative of the severity or prognosis of an alpha-synuclein related disease, PD, or both, in the subject.

As used herein, the term “predetermined calibrating control” refers to a gradual scale comprising the levels of at least one phosphoinositide according to which a subject can be classified in the context of the severity or prognosis of an alpha-synuclein related disease, PD, or both.

In some embodiments, a calibrating control characterized by at least: 1%, 2%, 3%, 4%, 5%, or 6% increased or decreased level in the amount of at least one phosphoinositide compared to a baseline, or any value and range therebetween, is indicative of low to mild severity of an alpha-synuclein related disease, PD, or both.

In some embodiments, a calibrating control characterized by at least: 6.1%, 7%, 8%, 9%, 10%, or 11% increased or decreased level in the amount of at least one phosphoinositide compared to a baseline, or any value and range therebetween, is indicative of mild to high severity of an alpha-synuclein related disease, PD, or both.

In some embodiments, a calibrating control characterized by at least: 11.1%, 12%, 13%, 15%, 17%, 19%, or 20% increased level in the amount of at least one phosphoinositide compared to a baseline, or any value and range therebetween, is indicative of high severity of an alpha-synuclein related disease, PD, or both.

In some embodiments, a baseline or a standard level of at least one phosphoinositide in blood, such as PI4,5P2, is at least 132 ng/μl blood/total phosphates, at least 134 ng/μl blood/total phosphates, at least 136 ng/μl blood/total phosphates, at least 138 ng/μl blood/total phosphates, at least 140 ng/μl blood/total phosphates, at least 142 ng/μl blood/total phosphates, at least 144 ng/μl blood/total phosphates, at least 146 ng/μl blood/total phosphates, at least 148 ng/μl blood/total phosphates, at least 150 ng/μl blood/total phosphates, at least 155 ng/μl blood/total phosphates, at least 160 ng/μl blood/total phosphates, at least 165 ng/μl blood/total phosphates, or at least 170 ng/μl blood/total phosphates, or any value and range therebetween. Each possibility represents a separate embodiment of the invention. In some embodiments, a baseline or standard level of at least one phosphoinositide in blood, such as PI4,5P2, is 132 to 140 ng/μl blood/total phosphates, 136 to 145 ng/μl blood/total phosphates, 140 to 155 ng/μl blood/total phosphates, 135 to 160 ng/μl blood/total phosphates, or 137 to 165 ng/μl blood/total phosphates. Each possibility represents a separate embodiment of the invention.

In some embodiments, a baseline or a standard level of at least one phosphoinositide in blood, such as PI4,5P2, is at least 6.45 ng/ml blood/total phosphates, at least 6.5 ng/ml blood/total phosphates, at least 6.9 ng/ml blood/total phosphates, at least 7.0 ng/ml blood/total phosphates, at least 7.1 ng/ml blood/total phosphates, 7.2 ng/ml blood/total phosphates, 7.4 ng/ml blood/total phosphates, 7.6 ng/ml blood/total phosphates, 7.8 ng/ml blood/total phosphates, or at least 8.0 ng/ml blood/total phosphates, or any value and range therebetween. Each possibility represents a separate embodiment of the invention. In some embodiments, a baseline or standard level of at least one phosphoinositide in blood, such as PI4,5P2, is 6.42 to 8.5 ng/ml blood/total phosphates, 6.45 to 9.5 ng/ml blood/total phosphates, 6.5 to 7.5 ng/ml blood/total phosphates, 7.0 to 8.0 ng/ml blood/total phosphates, or 6.7 to 8.2 ng/ml blood/total phosphates. Each possibility represents a separate embodiment of the invention.

In some embodiments, a baseline or a standard level is an average baseline or an average standard level. In some embodiments, average is or refers to an average value of a healthy population.

In some embodiments, there is provided a method for monitoring the responsiveness of a subject to anti alpha-synuclein related disease therapy, PD therapy, or both. In some embodiments, the monitoring comprises: determining an amount of at least one phosphoinositide in a sample obtained or derived from a subject, quantifying a level of decrease or increase in the amount of the at least one phosphoinositide compared to a predetermined calibrating control, or both.

In some embodiments, the method comprises: determining whether at least one phosphoinositide is present in an amount: (i) decreased below a baseline; or (ii) increased above a baseline, in a sample obtained or derived from a subject; and administering to the subject determined as having decreased or increased amount of the at least one phosphoinositide below or above the baseline, respectively, a therapeutically effective amount of pharmaceutical composition comprising an agent suitable for inhibiting or reducing alpha synuclein activity, pathogenicity, or both, anti-PD therapy, or both.

As used herein, the term “alpha-synuclein activity” refers to any pathogenic effect induced by, enhanced by, propagated by, involving, or any combination thereof, alpha-synuclein protein. In some embodiments, alpha synuclein is a mutated alpha synuclein. In some embodiments, alpha-synuclein activity, pathogenicity, or both, comprises alpha-synuclein aggregation. In some embodiments, alpha-synuclein activity, pathogenicity, or both, comprises a cellular disorder selected from: microtubule impairment, synaptic and mitochondrial dysfunctions, oxidative stress, dysregulation of calcium signaling, proteasomal pathway, lysosomal pathway, or any combination thereof.

In some embodiments, the method further comprising a monitoring step comprising at least once determining the amount of at least one phosphoinositide in sample obtained or derived from an administered or treated subject. In some embodiments, the monitoring step proceeds the administering step, as disclosed herein. In some embodiments, the monitoring step is performed at least once. In some embodiments, the monitoring step is performed multiple times. In some embodiments, the monitoring step is performed at least: once a week, once a month, once in 6 months, once a year, or any value and range therebetween. Each possibility represents a separate embodiment of the invention.

In some embodiments, the monitoring step is performed once a week to once a month, once in 2 months to 6 months, once in 3 months to 9 months, once in 4 months to 11 months, once in 12 months to 26 months. Each possibility represents a separate embodiment of the invention.

In some embodiments, the method comprises administering to a subject a therapeutically effective amount of a pharmaceutical composition comprising an agent characterized by being capable of modifying any one of: non-steady state levels of PI4,5P2, PI4,5P2-related signaling, and both.

As used herein, the term “steady state” represents the level of at least one phosphoinositide in a biological sample or tissue. In some embodiments, “steady state” represents the level of at least one phosphoinositide in blood. In some embodiments, blood comprises blood of a human subject.

As used herein, the term “modifying” comprises increasing or decreasing.

In some embodiments, modifying, e.g., increasing or decreasing, is compared to a non-treated control subject including a sample derived or obtained therefrom.

In some embodiments, the method comprises increasing or decreasing: the non-steady state levels of PI4,5P2 to physiological steady state levels, PI4,5P2-related signaling, or both, in the subject.

In some embodiments, the method comprises rectifying non-steady state levels of PI4.5P2.

As used herein, the term “physiological steady-state levels of PI4,5P2” refers to the levels of PI4,5P2 determined in a healthy subject, or any sample derived or obtained therefrom. In some embodiments, “physiological steady-state levels of PI4,5P2” refers to the levels of PI4,5P2 determined in a subject not afflicted with or not having increased risk of developing an alpha-synuclein related disease, PD, or both.

According to some embodiments, there is provided a method for determining a subject is in need of a treatment with an agent characterized by being capable of decreasing signaling transduced by a member of PI4,5P2 signal transduction pathway.

As used herein, the term “PI4,5P2 signal transduction pathway” refers to a signal transduction pathway which involves phosphatidylinositol 4,5-bisphosphate or PtdIns(4,5)P2 (PIP2 or PI(4,5)P2). Phosphatidylinositol is a minor phospholipid component of the cell membrane. Phosphatidylinositol(s) serve as substrates for diverse signaling proteins and their respective cascades, and are abundant at the plasma membrane. In some embodiments, PI4,5P2 signal transduction pathway or signaling pathways, comprise: PIP2 cycle, PI3K signaling, PI5P metabolism, or any combination thereof.

The PI3K/AKT pathway is activated in brains affected with PD and models thereof. Activated PI3K/AKT is exemplified by increases in PI3-Ps, including PI3,4,5P3 and PI3,4P2 that are required for the phosphorylation and activation of AKT phosphorylation at Thr308 and Ser 473. α-Syn is involved in activation of PI3K/AKT pathway. PI3K/AKT activation by α-Syn involves protein palmitoylation. Treatments in disease models with inhibitors of the PI3K/AKT pathway, including LY294002, PIK75, TGX22 and MK2206, reduce the aberrant activation of this pathway by α-Syn. Inhibitors of the PI3K/AKT pathway are useful to inhibit α-Syn′ toxicity. These may include Alpelisib and additional inhibitors of PI3K class I that act peripherally not crossing the blood brain barrier (BBB); or GDC-0084 and similar agents that can cross the BBB and act at the central nervous system. Or inhibitors of class II PI3K, for example, HY-151491, HY-151492 or HY-151490.α-Syn enhances PI3K activity by enhancing protein palmitoylation, including Ras palmitoylation that regulates PI3K activity. Class I and class II PI3Ks enzymes harbor a Ras binding domain and thus are affected by α-Syn. Class I and class II PI3Ks enzymes are activated by α-Syn and are targets for PD therapy. Inhibitors for class I and class II PI3Ks may be used for drug repurposing

According to some embodiments, there is provided a method for ameliorating or treating a subject afflicted with or at increased risk of developing an alpha-synuclein-associated disease or disorder, PD, or both.

In some embodiments, the treating comprises administering to the subject a therapeutically effective amount of a PI3K/AKT/mTORC2 signaling inhibitor, or a pharmaceutical composition comprising same.

In some embodiments, treating comprises gene therapy.

In some embodiments, gene therapy comprises CRISPR-Cas system. In some embodiments, Cas comprises Cas 9.

In some embodiments, the method comprises administering to the subject an inhibitor being a CRISPR-Cas system, configured to reduce or inhibit signaling of the PI3K/AKT/mTORC2 pathway.

In some embodiments, reducing or inhibiting is via gene silencing, e.g., RNAi. In some embodiments, gene silencing comprises knockdown of a transcript encoded by at least one gene of the PI3K/AKT/mTORC2 pathway.

In some embodiments, reducing or inhibiting is via gene knockout.

In some embodiments, reducing or inhibiting is via gene knock in, e.g., resulting in mistranslation, frameshift, premature stop codon, or any combination thereof, resulting in translation of an unopenable, non-functional, or absent protein of the PI3K/AKT/mTORC2 pathway.

In some embodiments, the PI3K/AKT/mTORC2 signaling inhibitor comprises an inhibitor suitable for treating of a cell proliferation related disease, e.g., cancer.

In some embodiments, the PI3K/AKT/mTORC2 signaling inhibitor comprises an inhibitor configured to cross the blood brain barrier (BBB). In some embodiments, the PI3K/AKT/mTORC2 signaling inhibitor comprises an inhibitor characterized by being capable of crossing the brain blood barrier (BBB).

In some embodiments, the inhibitor comprises an inhibitor being uncapable of crossing the BBB.

In some embodiments, a PI3K/AKT/mTORC2 signaling inhibitor comprises Alpelisib. In some embodiments, a PI3K/AKT/mTORC2 signaling inhibitor comprises GDC-0084

In some embodiments, a PI3K/AKT/mTORC2 signaling inhibitor comprises: HY-151491, HY-151492, HY-151490, or any combination thereof.

As used herein, the terms “alpha-synuclein-associated disease or disorder” or “synucleopathies” are interchangeable, and refer to any disease or disorder (including any symptom associated therewith) involving alpha-synuclein as part of the disease or disorder pathogenesis or pathophysiology.

In some embodiments, an alpha-synuclein associated or related disease or disorder is selected from: Parkinson's disease (PD), PD with dementia (PDD), Lewy body dementia, Alzheimer's disease (AD), multiple system atrophy, NIEMANN-PICK-type A, or any combination thereof.

In some embodiments, PD comprises PD with dementia (PDD).

As used herein, the terms “Parkinson's disease” or “PD” refer to a slowly progressive, degenerative disorder characterized by resting tremor, stiffness (rigidity), slow and decreased movement (bradykinesia), and eventually gait and/or postural instability.

As used herein, the term “anti-PD therapy” includes any drug suitable for treating PD which would be known for one of ordinary skill in the art.

In some embodiments, an anti-PD therapy comprises: a dopamine precursor, an antiviral drug, a dopamine agonist, an anticholinergic drug. a monoamine osidase type B (AMO-B) inhibitor, a catechol O-methyltransferase (COMT) inhibitor, or any plurality and/or combination thereof.

In some embodiments, anti-PD therapy is selected from: Carbidopa (or levodopa), pramipexole, ropinirole, benztropine, trihexyphenidyl, rasagiline, selegiline, entacapone, opicapone, tolcapone, or any combination thereof.

In some embodiments, the method comprises determining an amount of at least one phosphoinositide in a sample obtained or derived from a subject.

In some embodiments, the phosphoinositide is selected from: PI4,5P2, PI, PI4P, PI3,4,5P3, or any combination thereof.

Methods for determining an amount of phosphoinositide(s) in a sample are common and would be apparent to one of ordinary skill in the art of biochemistry and cell biology. A non-limiting example for such determination methods includes, but is not limited to, an enzyme-linked immunosorbent assay (ELISA) utilizing antibodies having specific binding affinity to a phosphoinositide of desire, such as exemplified herein.

In some embodiments, a decrease in the amount of the at least one phosphoinositide below a pre-determined threshold is indicative of the subject being in need of a treatment with the agent characterized by being capable of decreasing signaling transduced by a member of PI4,5P2 signal transduction pathway.

In some embodiments, any one of an increase and a lack of a decrease in the amount of the at least one phosphoinositide below a pre-determined threshold is indicative of the subject not being in need of a treatment with the agent characterized by being capable of decreasing signaling transduced by a member of PI4,5P2 signal transduction pathway.

In some embodiments, a member of the PI4,5P2 signal transduction pathway is selected from: PI4,5P2, PI, PI4P, PI3,4,5P3, or any combination thereof.

In some embodiments, the method further comprises determining at least one biomarker selected from: total α-Syn, PKres α-Syn, phosphorylated Ser 129 α-Syn, oligomeric α-Syn, iron, or any combination thereof, in a sample obtained or derived from a subject.

In some embodiments, a sample comprises any biological sample obtained or derived from a subject.

In some embodiments, the sample is selected from: whole blood or any fraction thereof, erythrocytes, platelets, saliva, septum, tears, feces, or any combination thereof.

In some embodiments, the sample comprises blood. In some embodiments, the sample comprises or is a blood sample obtained or derived from a subject.

In some embodiments, the sample comprises blood cells. In some embodiments, the sample comprises an extract of blood cells. In some embodiments, the extract comprises a lipids. In some embodiments, the extract comprises polar lipids. In some embodiments, the extract comprises a lipids of blood cells. In some embodiments, the extract comprises polar lipids of blood cells.

In some embodiments, the method further comprises step comprising administering to the subject determined to be in need of a treatment, as described herein, a therapeutically effective amount of an agent characterized by being capable of decreasing signaling transduced by a member of PI4,5P2 signal transduction pathway.

In some embodiments, the subject is prodromal or pre-symptomatic to an alpha-synuclein related disease.

In some embodiments, the subject is being treated for an alpha-synuclein related disease, PD, or both.

In some embodiments, the subject is in need of ameliorating or treating of an alpha-synuclein associated or related disease or disorder, PD, or both.

In some embodiments, the subject is afflicted with or at increased risk of developing an alpha-synuclein-associated or related disease or disorder, PD, or both.

In some embodiments, the subject is a human subject.

In some embodiments, increased risk refers to having increased predisposition to developing a disease or disorder as described herein.

In some embodiments, “increase” or “increasing” is by at least 5%, 15%, 25%, 40%, 50%, 75%, 100%, 250%, 500%, 750%, 1,000%, or any value and range therebetween, increase. Each possibility represents a separate embodiment of the invention.

In some embodiments, “increase” or “increasing” is by 5-100%, 15-250%, 25-600%, 40-900%, 50-1,100%, 1-75%, 10-450%, 25-375%, 50-680%, 75-400%, or 100-1,000% increase. Each possibility represents a separate embodiment of the invention.

In some embodiments, “decrease” or “decreasing” is by at least 5%, 15%, 25%, 40%, 50%, 75%, or 100%, or any value and range therebetween, decrease. Each possibility represents a separate embodiment of the invention.

In some embodiments, “decrease” or “decreasing” is by 5-10%, 15-50%, 20-65%, 40-90%, 50-100%, 1-70%, 10-90%, 25-75%, 5-85%, or 1-99% decrease. Each possibility represents a separate embodiment of the invention.

In some embodiments, any one of “increase” or “increasing” and “decrease” or “decreasing” is compared to a control.

In some embodiments, a control comprises a control subject or a sample derived or obtained therefrom. In some embodiments, a control comprises a healthy subject or a sample derived or obtained therefrom. In some embodiments, a control comprises a subject not afflicted with an alpha-synuclein-related disease or disorder, PD, or both, or a sample derived or obtained therefrom. In some embodiments, a control comprises the subject to be treated according to the herein disclosed method before being diagnosed or developing an alpha-synuclein-related disease or disorder, PD, or both, or a sample derived or obtained therefrom.

As used herein, the term “predisposition” refers to the susceptibility of a subject to a disease, such as an alpha-synuclein-related disease or disorder, PD, or both. In some embodiments, detecting or determining predisposition comprises detecting or determining the presence of the disease itself. In some embodiments, detecting or determining a predisposition comprises any one of: detecting or determining the risk of developing the disease, determining the susceptibility of the subject to developing the disease, having a poor prognosis for the disease, or any combination thereof. In some embodiments, a subject having a predisposition to a disease is at risk of developing the disease.

In some embodiments, the agent decreases any one of: an amount of the member of PI4,5P2 signal transduction pathway, an amount of an active form of the member of PI4,5P2 signal transduction pathway, an amount of membrane associated form of the member of PI4,5P2 signal transduction pathway, or any combination thereof, in the subject.

In some embodiments, decreasing the amount of the member comprises any one of: decreasing the production rate of the member, increasing the degradation rate of the member, or both, in the subject.

In some embodiments, decreasing the production rate of the member comprises decreasing any one of: phosphatidylinositol synthesis, PI4-kinase activity, PI5-kinase activity, or any combination thereof. In some embodiments, decreasing the degradation rate of the member comprises contacting the member with a phosphatase characterized by being capable of removing a phosphate group at position PI4, position PI5, or both of the member. In some embodiments, decreasing comprises: decreasing both the production rate of the member comprises decreasing any one of: phosphatidylinositol synthesis, PI4-kinase activity, PI5-kinase activity, and contacting the member with a phosphatase characterized by being capable of removing a phosphate group at position PI4, position PI5, or both of the member.

In some embodiments, decreasing the amount of membrane associated form of the member comprises decreasing translocation, migration, or both, of the member to a cell membrane. In some embodiments, decreasing the amount of membrane associated form of the member comprises decreasing translocation, migration, or both, of the member to the peripheral membrane of a cell of a subject. In some embodiments, the cell is a neural cell. In some embodiments, the cell is a neuron. In some embodiments, the neuron is a cortical neuron.

In some embodiments, decreasing the amount of an active form of the member comprises sequestering the active form of the member.

In some embodiments, decreasing translocation of the member to a cell membrane comprises reducing or inhibiting activity of one or more proteins characterized by being capable of binding phosphatidylinositol, transferring phosphatidylinositol to the cell membrane, or both.

In some embodiments, sequestering comprises contacting the member with an antagonist. In some embodiments, sequestering comprises administering to the subject a therapeutically effective amount of an antagonist having specific binding affinity to the member, as disclosed herein.

In some embodiments, the antagonist comprises any one of: an antibody, a binding protein or a binding domain thereof, a soluble receptor, or any functional fragment thereof.

In some embodiments, a functional fragment, as used herein, is characterized by having increased binding affinity to the member. In some embodiments, a functional fragment, as used herein, is characterized by having specific binding affinity to the member.

In some embodiments, ameliorating or treating comprises: improving at least one clinical manifestation associated with the disease or disorder pathology, decreasing the rate of functional deterioration, decreasing the rate, postponing, or preventing conversion to a full disease of a subject pre-disposed to developing the disease later in life, or any combination thereof.

In some embodiments, the member of PI4,5P2 signal transduction pathway comprises or consists of PI4P.

In some embodiments, the agent characterized by being capable of decreasing signaling transduced by PI4P levels is 3-(4-carbamoylphenyl)-N-(5-cyanopyridin-2-yl)-N-methylpyrazolo[1,5-a]pyridine-5-carboxamide, 5-2-amino-1-(4-morpholinophenyl)-1H-benzo[d]imidazol-6-yl)-N-(2-fluorophenyl)-2-methoxypyridine-3-sulfonamide, or a combination thereof.

In some embodiments, 3-(4-carbamoylphenyl)-N-(5-cyanopyridin-2-yl)-N-methylpyrazolo[1,5-a]pyridine-5-carboxamide (also known as KDU731, Chemical abstract CID 76281701, Synthesis of this compound is detailed in U.S. Pat. Nos. 9,556,169 and 8,871,754 incorporated herein by reference) is represented by the formula:

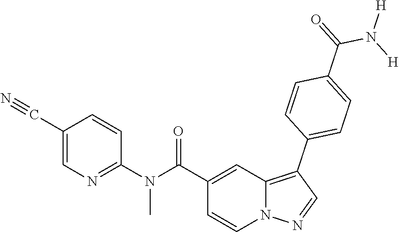 [SMILES: CN(C(=O)c1ccn2ncc(-c3ccc(C(N)=O)cc3)c2c1)c1ccc(C#N)cn1]

In some embodiments, 5-2-amino-1-(4-morpholinophenyl)-1H-benzo[d]imidazol-6-yl)-N-(2-fluorophenyl)-2-methoxypyridine-3-sulfonamide (CAS 1416334-69-4), is represented by the formula:

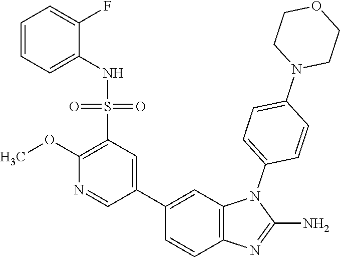 [SMILES: COc1ncc(-c2ccc3nc(N)n(-c4ccc(N5CCOCC5)cc4)c3c2)cc1S(=O)(=O)Nc1ccccc1F]

In some embodiments, determining an amount of the at least one phosphoinositide comprises contacting the sample obtained or derived from the subject with a phosphoinositide antagonist having specific binding affinity to: PI3P, PI4P, PI5P, PI4,5P2, PI3,4P2, PI3,5P2, PI3,4,5P3, or any combination thereof.

In some embodiments, determining an amount of the at least one phosphoinositide comprises contacting the sample obtained or derived from the subject with at least one phosphoinositide antagonist having specific binding affinity to: PI3P, PI4P, PI5P, PI4,5P2, PI3,4P2, PI3,5P2, PI3,4,5P3, or any combination thereof.

In some embodiments, determining an amount of the at least one phosphoinositide comprises contacting the sample obtained or derived from the subject with a plurality of phosphoinositide antagonist having specific binding affinity to: PI3P, PI4P, PI5P, PI4,5P2, PI3,4P2, PI3,5P2, PI3,4,5P3, or any combination thereof.

In some embodiments, the antagonist has specific binding affinity to PI4,5P2.

In some embodiments, the antagonist comprises: an antibody, a phosphoinositide binding protein or a binding domain thereof, a soluble receptor, or any functional fragment thereof.

### A Kit

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

According to some embodiments, there is provided a kit for diagnosing, prognosing, or both, an alpha-synuclein related disease, PD, or both, in a subject.

In some embodiments, the kit is for determining the amount, level, concentration, or any combination thereof, of at least one phosphoinositide in a sample obtained or derived form a subject.

In some embodiments, the kit is for determining the suitability of a subject to anti alpha-synuclein related disease therapy.

In some embodiments, the kit comprises: at least one phosphoinositide antagonist, and: a baseline control as disclosed herein, a calibrating control as disclosed herein, or both.

In some embodiments, the kit further comprises instructions for determining an amount of the at least one phosphoinositide, in a sample obtained or derived from a subject.

In some embodiments, the kit further comprises instructions for quantifying a level of decrease or increase in the amount of the at least one phosphoinositide compared to a calibrating control.

In some embodiments, the kit further comprises instructions for ameliorating or treating a subject diagnosed or prognosed for an alpha-synuclein related disease, PD, or both, as disclosed herein, the instructions comprise administering to the diagnosed or prognosed subject a therapeutically effective amount of: an agent characterized by being capable of inhibiting or reducing alpha synuclein activity, pathogenicity, an anti-PD therapy (as described herein) or both, a in the diagnosed or prognosed subject.

In some embodiments, the kit further comprises at least one phosphoinositide antagonist suitable for administration to a subject diagnosed or prognosed for an alpha-synuclein related disease, as disclosed herein.

In some embodiments, the at least one phosphoinositide antagonist used for diagnosing, prognosing, or both, an alpha-synuclein related disease in a subject and/or for determining the amount, level, concentration, or any combination thereof, of at least one phosphoinositide in a sample obtained or derived form a subject, and the at least one phosphoinositide antagonist used ameliorating or treating a subject diagnosed or prognosed for an alpha-synuclein related disease, PD, or both, are different. In some embodiments, the at least one phosphoinositide antagonist used for diagnosing, prognosing, or both, an alpha-synuclein related disease, PD, or both, in a subject and/or for determining the amount, level, concentration, or any combination thereof, of at least one phosphoinositide in a sample obtained or derived form a subject, and the at least one phosphoinositide antagonist used ameliorating or treating a subject diagnosed or prognosed for an alpha-synuclein related disease, PD, or both, are the same.

In some embodiments, the kit is for monitoring any one of: disease progression or regression, responsiveness of a subject to therapy, or both, of an alpha-synuclein related disease, PD, or both.

In some embodiments, the kit comprises at least one phosphoinositide antagonist.

In some embodiments, the kit further comprises instructions for determining an amount of the at least one phosphoinositide. In some embodiments the determining is in a sample obtained or derived from a subject. In some embodiments, the determining, as disclosed herein for the kit of the invention, and/or the method of the invention, comprises in vitro or ex vivo determining. In some embodiments, the determining is not in a subject's body.

It would be apparent to one of ordinary skill in the art that in vitro and/or ex vivo are performed outside a living organism, e.g., such as in a plate.

In some embodiments, ex vivo and/or in vitro is in a tube (e.g., a plastic tube, such as an Eppendorf tube), a plate, a well plate, or any other compatible surface and/or vessel/container, for such an assay which would be apparent to one of ordinary skill in the art.

In some embodiments, the kit further comprises instructions for determining an amount of: total α-Syn; PK^{res} α-Syn, Phosphorylated Ser 129 α-Syn, iron, or any combination thereof, in a sample obtained or derived from a subject.

In some embodiments, any one of: (a) at least one phosphoinositide; (b) total α-Syn; pK^{res} α-syn, Phosphorylated Ser 129 α-Syn, iron, or any combination thereof; and (c) a combination of (a) and (b), in an amount below a predetermined threshold, is indicative of the subject being suitable for alpha-synuclein-associated disease or disorder therapy.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value (±10%). For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Human Brains

Formalin fixed, paraffin embedded brain sections, containing the caudate and internal capsule, of advanced PD (Braak stage 5-6) and age-matched control brains, were supplied by the Multiple Sclerosis Society Tissue Bank, funded by the Multiple Sclerosis Society of Great Britain and Northern Ireland, registered charity 207495. Additional brain sections of early PD cases (Unified stage IIa-IIb) and relevant control brains were provided by the Banner Sun Health Research Institute, Sun City, Arizona, USA (Table 1). The approval for the use of human tissue material was obtained from the Peer Review Panel of the Parkinson's UK Brain Bank and the Brain and Body Donation Program at Sun Health Research Institute; the latter's operations are approved by the Western Institutional Review Board (Seattle, Wash.).

TABLE 1
Post-mortem human brains

| | | | | | | | |
|---|---|---|---|---|---|---|---|
| | | | | | | | Braak/ |
| | | | | age at | | PMl | Unified |
| source | case ID | gender | age | diagnosis | duration | (h) | stage |

PD

| | | | | | | | |
|---|---|---|---|---|---|---|---|
| Banner Sun | 99-09 | F | 77 | 56 | 21 | 2.66 | unified IIa |
| Health | 05-17 | F | 84 | | 22 | 2 | unified IIa |
| Research | 04-17 | M | 83 | 75 | 8 | 2 | unified IIa |
| Institute | 00-09 | M | 64 | 49 | 15 | 4 | unified IIb |
| | 00-24 | M | 72 | 55 | 17 | 10 | unified IIb |
| UK brain | PD084 | M | 86 | 78 | 9 | 3 | Braak 5 |
| bank | PD093 | F | 81 | 67 | 14 | 22 | Braak 6 |
| | PD099 | M | 82 | 72 | 11 | 10 | Braak 6 |
| | PD104 | M | 75 | 50 | 25 | 15 | Braak 5 |
| | PD109 | M | 72 | 66 | 6 | 9 | Braak 4 |
| | PD117 | F | 77 | 46 | 31 | 6 | Braak 5 |

Control

| | | | | | |
|---|---|---|---|---|---|
| Banner Sun | 09-57 | M | 80 | n.a. | 3.5 |
| Health | 10-13 | M | 85 | n.a. | 3 |
| Research | 15-60 | M | 82 | n.a. | 3.33 |
| Institute | | | | | |
| UK brain | PDC094 | F | 80 | | 63 |
| bank | C022 | F | 69 | | 33 |
| | C032 | M | 88 | | 22 |
| | C045 | M | 77 | | 22 |

PMI, post-mortem interval in hours (h).

### Mice

The human PrP-A53T α-Syn tg mouse line was purchased from Jackson Laboratory (Bar Harbor, Me., USA) as hemizygous; cross-bred with α-Syn^{-/-} C57BL/6JOlaHsd mice (Harlan Laboratories, Jerusalem, Israel) to silence endogenous mouse α-Syn; and then bred to achieve homozygosity of the human A53T α-Syn transgene. α-Syn^{-/-} C57BL/6JOlaHsd genotype was used as control mice. The PrP-A53T α-Syn tg model was shown in previous studies to develop motor disabilities and to accumulate α-Syn pathology in an age-dependent manner. That is, mice appear generally healthy and show no evidence of α-Syn pathology up to the age of 8-9 months. However, at 12 months of age and older, the large majority of mice in the colony show signs of motor disabilities accompanied with pathogenic accumulations of α-Syn in the central nervous system. The number of sick mice grow with age and the oldest mice in the colony are ˜16 months old. All animal welfare and experimental protocols were approved by the Committee for the Ethics of Animal Experiments of the Hebrew University of Jerusalem NIH approval #OPRR-A01-5011 (Permit number: MD-16-14826-3).

Thy-1 hWT α-Syn mice were obtained from Prof. Eliezer Masliah (UCSD, USA). Control mice were non-transgenic littermates. The Thy-1 hWT α-Syn mice show early signs of learning and motor disabilities at 2-4 months of age, which worsen at 8-10 months of age. α-Syn pathology for the Thy-1 hWT α-Syn mice was demonstrated at 8-12 months of age.

5XFAD mouse model shows accumulation of amyloid pathology, starting at the age of 4 months, in addition to cognitive impairment, starting at 6 months of age.

Mice were housed at a 12-hour dark/light cycle and were allowed free access to food and water. This study was carried out in strict accordance with the recommendations in the Guide for the Care and Use of Laboratory Animals of the National Institutes of Health. Adequate measures were taken to minimize pain and suffering.

### Transmission Electron Microscopy

Mice were anesthetized and perfused in Karnovsky's fixative solution (2% formaldehyde and 2.5% glutaraldehyde in 0.1 M Sodium Cacodylate buffer, pH 7.4). Mouse brains were removed and 100 μm coronal sections were obtained using a vibratome (Leica Biosystems, IL, USA). Brain sections were fixed in Karnovsky's fixative solution for two hours at room temperature and then transferred to 4° C. for an additional 24 hours. Sections were washed four times with 0.1 M sodium cacodylate buffer (pH 7.3) and incubated for 1 hour in 1% osmium tetroxide, 1.5% potassium ferricyanide in sodium cacodylate. Sections were then washed 4 times in the same buffer; dehydrated with graded series of ethanol solutions (30, 50, 70, 80, 90, 95%) for 10 minutes each; then in 100% ethanol 3 times for 20 minutes each; followed by two changes of propylene oxide. Brain sections were infiltrated with series of epoxy resin, (25, 50, 75, 100%) for 24 hours each and polymerized in the oven at 60° C. for 48 hours. The blocks were sectioned by an ultramicrotome (Ultracut E, Riechert-Jung, Ontario, Canada) and sections of 80 nm were stained with uranyl acetate and lead citrate. Sections were observed using a Jeol JEM 1400 Plus Transmission Electron Microscope and pictures were taken using a Gatan Orius CCD camera.

### Cell Cultures

HEK 293T, HeLa and SH-SYSY, and an inducible α-Syn-expressing SH-SYSY cell lines were maintained in Dulbecco's modified eagle's medium (DMEM) supplemented with 10% FBS; 2% L-glutamine; 1% penicillin/streptomycin, sodium-pyruvate and non-essential amino acids (Biological Industries, Beit-Haemek, Israel). α-Syn expression was induced in the inducible SH-SY5Y cell line with 1 μg/ml doxycycline (Dox, Sigma-Aldrich, Rehovot, Israel). SK-Mel2 cells express detectable levels of endogenous α-Syn, however, these are lowered with passages. Thus, a large number of aliquots at passage twelve were kept frozen and experiments were performed between weeks 2-6 from thawing a frozen aliquot. SK-Mel2 cells were maintained in minimum essential medium (MEM; Sigma-Aldrich, Rehovot, Israel) supplemented with 10% FBS; 1% L-glutamine, penicillin/streptomycin, and sodium-pyruvate. Cultures were maintained at 37° C. in a 95% air/5% CO₂ humidified incubator.

### Plasmids

Costume-ready Mission shRNA were from Sigma-Aldrich. Including, shSNCA (TRCN0000272292), shCntrl and shNIR2 (TRCN0000029763), that was successfully used previously; pGFP-C1-PLCδ1-PH (Addgene #21179 from Tobias Meyer); pEGFPC1-Sj-1-170 and GFP-PIPKIγ (Addgene #22294 and #22299 from Pietro De Camilli); pFSy(1.1)GW (Addgene #27232 from Pavel Osten). CFP-FKBP-PIPK and Lyn-FRB. mCherry-NIR2. pFSy-α-Syn was constructed by ligation of a full-length α-Syn cDNA, amplified by PCR, with pFSy(1.1)GW, following digestion with AgeI-HF/Xba-I restriction enzymes. The following primers were used for α-Syn amplification: forward: 5′-GAATCACCGGTGCCGCCACCATGGATGTATTCATGAAAGG-3′ (SEQ ID NO: 1) and reverse: 5′-TAACTCTAGATTAGGCTTCAGGTTCGTAGT-3′ (SEQ ID NO: 2).

In addition, an adenovirus expressing sypHy under the human synapsin (hSyn) promotor (AAV1/2 hSyn:Synaptophysin-2XpHluorin) and AAV1/2 hSyn:WT α-Syn (54) were used. AAV1/2 hSyn:mutant α-Syn were constructed by PCR amplification followed by complete sequencing of the inserts.

### Viral Production and Transduction

Lentiviral particles were produced by co-transfecting HEK 293T cells with a set of three plasmids: pCMVAR8.91; pMD2.G; and a transfer plasmid, either pFSy(1.1) or pLKO-1-puro. Transfection was performed in 10 cm dishes (2×10⁶ cells) using 50 μg polyethylenimine (PEI) incubated with 12.5 μg DNA at 1:1:1 molar ratio for the three plasmids. 3-4 days after transfection, the conditioning medium was collected and spun for 5 minutes at 1,500 g to remove cell debris, filtered through a 0.45 μm membrane and spun at 93,000 g for 2 hours, at 4° C. in a swinging-bucket rotor. Pellets containing virus particles were collected in serum-free medium and stored at −80° C., in aliquots. Each aliquot was thawed once, immediately before use. Virus titer was determined for each preparation following transduction of naïve SH-SY5Y cells, by quantitative PCR using specific primers either for WPRE gene (pFSy plasmids): forward 5′-CCGTTGTCAGGCAACGTG-3′ (SEQ ID NO: 3) and reverse 5′-AGCTGACAGGTGGTGGCAAT-3′ (SEQ ID NO: 4); or Puromycin resistance gene (pLKO-1-puro plasmids): forward, 5′-TCACCGAGCTGCAAGAACTCT-3′ (SEQ ID NO: 5) and reverse primer, 5′-CCCACACCTTGCCGATGT-3′ (SEQ ID NO: 6). Primer sequence for human SNCA: forward: 5′-GCAGGGAGCATTGCAGCAGC-3 (SEQ ID NO: 7) and reverse 5′-GGCTTCAGGTTCGTAGTCTTG-3′ (SEQ ID NO: 8); Nir2: forward: 5′-GCTTTGATGCACTCTGCCAC-3′ (SEQ ID NO: 9) and reverse: 5′-AGCTCATTGTTCATGCTCCC-3 (SEQ ID NO: 10); G6PD: forward: 5′-CACCATCTGGTGGCTGTTC-3 (SEQ ID NO: 11) and reverse 5′-TCACTCTGTTTGCGGATGTC-3 (SEQ ID NO: 12).

Viral transduction of cultured cells was performed by incubating the cells (1.5×10⁶) in FBS free-DMEM, containing viral particles and polybreane (4 μg/ml) for 6 hours. The conditioning medium was then replaced with 10% FBS-supplemented DMEM. Viral transduction of primary cortical neurons was performed at 1 day in vitro (DIV) in full Neurobasal-A medium without polybreane.

AAV1/2 particles were produced as previously described. Briefly, HEK293T cells were co-transfected with the pD1 and pD2 helper plasmids and a plasmid containing the cDNA of interest located between AAV2 ITRs, preceded by the hSyn promotor. After 3 days of incubation at 37° C. in a humidified 5% CO₂ incubator, cells were lysed in lysis solution (150 mM NaCl, 50 mM Tris-HCl pH 8.5) using 3 rapid freeze-thaw cycles (in an ethanol bath chilled to −80° C. and a heated 37° C. water bath). The supernatant was treated with 10 units/ml benzonaze (Sigma-Aldrich, Rehovot, Israel), cleared by centrifugation, and filtrated through a 0.45 μm membrane. The viral particles were maintained at 4° C. until use. Viral titer was determined by infecting neuronal cultures, aiming for 80-90% infection efficiency, verified by immunofluorescence or direct fluorescence imaging, as applicable. Viral titer was determined by adding 0.2-2 μl of the viral prep directly to the growth medium at 5 DIV.

### Primary Cultures

Cortical cultures were prepared from cortices, dissected from a day old (P1) C57BL/6J or C57BL/6JOlaHsd mouse brains, as described previously. Cells (˜50,000) were plated onto coverslips, pre-coated with 12.5 μg/ml poly-D-lysine (Sigma-Aldrich) in a 12-well dish. Cortical neurons were maintained in Neurobasal-A medium (Gibco, Thermo Fisher Scientific, Petah Tikva, Israel) and supplemented with 2% B-27 (Gibco, Thermo Fisher Scientific); 1% L-glutamine; 0.5% penicillin/streptomycin. To eliminate glia cells, 1 μM cytosine β-D-arabinofuranoside (Ara-C; Sigma-Aldrich) was added to the culture at 1-2 DIV. Culture medium was partially (25-50%) replaced every 4 days. Cultures were maintained at 37° C. in a 5% CO₂ humidified incubator.

Hippocampal cultures were prepared from CA1-CA3 regions dissected from a day old (P1) WT C57BL/6J or α-Syn^{-/-} C57BL/6JOlaHsd mouse brains as described previously.

Mesencephalic neurons were prepared from brains of mice at E13.5 embryos as described. Electroporation of primary neurons was performed on day of preparation. Neurons were electroporated using the Amaxa Nucleofector (Lonza, Tuas, Singapore) according to manufacturer's protocol. 1×10⁶ cells were suspended in 100 μl of Ingenio electroporation solution (Minis Bio LLC, Madison, Wis., USA) containing 2.5 μg of DNA, in a nucleofection cuvette using program O-05. Cells were centrifuges to remove the electroporation medium and suspended in conditioning medium.

Primary hippocampal cultures were prepared by dissecting both hippocampi of P0-P1 C57BL/6JOlaHsd (α-Syn^{-/-}) mouse brains, as described previously (80,81). After trituration in 20 units/ml papain solution (Worthington Lakewood, N.J., USA), 1×10⁵ cells were plated on coverslips that were pre-coated with 5 μg/ml poly-D-lysine (Sigma-Aldrich, Rehovot, Israel) in a 24-well plate containing 1.5 ml Neurobasal-A medium (Gibco, Thermo Fisher Scientific, Petah Tikva, Israel) supplemented with 5% fetal bovine serum, 2% B-27 (Gibco, Thermo Fisher Scientific), 1% Glutamax I, and 1 μg/ml gentamicin. After 1 day, the solution was replaced to 1 ml Neurobasal-A supplemented with 2% B-27 and 1% Glutamax I. To slow the proliferation of glial cells, 1 μM cytosine β-D-arabinofuranoside (Ara-C; Sigma-Aldrich) was added to the culture at 2 days in vitro (DIV). Cultures were maintained at 37° C. in a 5% CO₂ humidified incubator until used at 8-13 DIV as indicated.

For measurements of Tf-568 endocytosis, neurons were prepared and grown as previously describe.

### Tissue Punches

Mouse brains were removed, washed with cold PBS, and placed on ice. A coronal segment of the brain, containing Bregma 0-(−3) was removed. Tissue punches (pooled from both hemispheres) were taken using a needle (1 mm) from the dorsal striatum. Punches were weighed and stored at −80° C. until use. Tissue was homogenized by ten up-and-down strokes of Teflon Dounce homogenizer, in 10 volumes (weight/volume) of homogenization buffer containing HEPES, 20 mM; EDTA, 1 mM; MgCl₂, 1 mM; sucrose, 0.32 M; a protease inhibitor cocktail (Sigma, Rehovot, Israel); and 1% NP-40, at 4° C. The homogenates were centrifuged at 1,500 g for 10 min to remove cell debris. Protein samples (30 μg) were loaded on a 10% SDS-PAGE, and following electrophoresis, were transferred to a nitrocellulose membrane (Biorad, Petach Tikva, Israel). The membrane was blocked with 10% non-fat dry milk in 10 mM Tris-HCl, 150 mM NaCl, pH 8.0, containing 0.1% Tween-20 (TBST) for 1 hour. The membrane was then incubated at 4° C. for 16-18 hours with the indicated antibody, in TBST. For antibody details see Table 2. Immunoreactive bands were detected with HRP-conjugated secondary antibody (1:10,000). The signal was visualized with EZ-ECL (Biological Industries, Beit Haemek, Israel), scanned by a Umax Magic Scan (Eastman Kodak, Rochester, N.Y., USA) and analyzed for density of each signal using UN-SCAN-IT GEL 3.1 software (Silk Scientific, Orem, UT, USA).

TABLE 2
List of antibodies

| | | | | Antigen |
|---|---|---|---|---|
| Antigen | Clone and source | Test | Dilution and blocking | retrieval |
| NF-200 | N4142, PC | IHC | 1:200 | 95° C., 20′ |
| | Sigma-Aldrich, | | o.n. 4° C., 10% NGS | |
| | Rehovot, Israel | ICC | 1:200 | |
| | | | 2 h RT, 1% BSA in PBS | |
| Non-P | SMI-32, MC | IHC | 1:1,000 | 110° C., 15′ |
| NF-H | Covance Inc., | | 2 h RT, 5% NGS | |
| | Princeton, NJ, USA | | | |
| P NF-H | SMI-31, MC | IHC | 1:1,000 | 110° C., 15′ |
| | Covance Inc., | | 2 h RT, 5% NGS | |
| | Princeton, NJ, USA | | | |
| APP | 22C11, MC | IHC | 1:2,000 | 110° C., 15′ |
| | Chemicon | | o.n. 4° C., CAS-block | |
| TH | TH-2, MC | IHC | 1:3,000 | 110° C., 15′ |
| | Sigma-Aldrich, | | o.n. 4° C., CAS-block | |
| | Rehovot, Israel | WB | 1:10,000 | |
| vGluT1 | AB#5905, PC | IHC | 1:1,000 | 110° C., 10′ |
| | Millipore | | 2 h 37° C., 10% NGS, | |
| | Rosh-Ha'ayin, Israel | | 0.3% Tx-100 | |
| PI4, 5P₂ | Z-P045, MC | IHC | 1:100 | 95° C., 10′ |
| | Echelon Biosciences | | o.n. 4° C. CAS-block | |
| | UT, USA | ICC | 1:200; 2 h RT 1% | |
| | | | BSA in PBS | |
| | | FACS | 1:200; 1.5 h 4° C., 1% | |
| | | | BSA in PBS | |
| α-tubulin | YL1/2 MC, | ICC | 1:1,000 2 h RT, 1% | |
| | Serotec | | BSA in PBS | |
| α-Syn | MJFR1, MC | ICC | 1:2,000; 2 h RT, 1% | |
| | ab138501, abcam | | BSA in PBS | |
| | | FACS | 1:2,000 1.5 h 4° C. 1% | |
| | | | BSA in PBS | |
| | | WB | 1:20,000 o.n 4° C. | |
| α-Syn | BD Transduction | IHC | 1:1000 | |
| | Labs | | | |
| α-Syn | C20, PC | ICC | 1:500 o.n 4° C. 1.5% | |
| | Santa Cruz. | | BSA in PBS | |
| α-Syn | Syn303, PC | IHC | 1:3,000; o.n 4° C. 1.5% | 95° C., 10′ |
| | Covance Inc., | | BSA in PBS | |
| | Princeton, NJ, USA | | | |
| Filament α-Syn | MJFR-14-6-4-2 | IHC | 1:3,000; 2.5 hours at | 110° C., 13′ |
| | Abcam | | RT, CAS-block | |
| Synaptophysin | Clone Z66, PC | WB | 1:250 | |
| | Thermo Sceintific | | | |
| DAT | 6-8D6 (sc-32259) | WB | 1:300 | |
| | Santa Cruz | | | |
| ß-actin | ac-15, MC, Sigma- | WB | 1:5,000 30 min RT | |
| | Aldrich | | | |
| GAP-43 | AB5220, PC | ICC | 1:1,000 2 h RT, 1% | |
| | Millipore | | BSA in PBS | |
| | Rosh-Ha'ayin, Israel | | | |
| Nir2 | AB22823 | WB | 1:5,000 90 min RT | |
| | Millipore | | | |
| | Rosh-Ha'ayin, Israel | | | |

* in 10 mM citrate buffer pH 6.0. PC, polyclonal; NDS, normal donkey serum; MC, monoclonal; NGS, Normal Goat serum, in Tris-Cl pH 7.3, 0.3% TX-100; CAS-block (Thermo Fisher) 10 min at RT; NDS, normal donkey serum (Jackson ImmunoResearch, ME, USA).

### Fluorescence Activated Cell Sorting (FACS)

Analysis was performed as previously described with some modifications. Cells were suspended and washed in clear DMEM; followed by 20 minutes fixation in 2% (v/w) paraformaldehyde at 4° C.; and permeabilization in 0.2% saponin in 1% BSA (w/v) for 15 minutes at 4° C. Cells were then incubated with anti α-Syn antibody (MJFR1, 1:2,000) and anti PI4,5P2 antibody (1:200, see Table 2) for 90 minutes at 4° C. with gentle rolling; washed and probed with the respective secondary antibody for 30 minutes at room temperature. FACS analysis in SK-mel2 cells was performed 7-14 days following viral transduction. During this time effective α-Syn knockdown was confirmed using rt-PCR and Western blotting. HEK 293T cells were analyzed by FACS forty-eight hours from transfection. Analyses were performed using BD LSRFortessa Cell Analyzer, equipped with 5 lasers (355, 405, 488, 561 and 640 nm) and the FLOWJO, LLC software. Mock-GFP, Sj-1 or PIPKIγ expression were directly detected at 488 nm based on a GFP-tag. Each experiment also included adequate compensation controls. In each experiment a control, consisting of cells grown and processed in parallel, treated with ionomycin (10 μM) for 5-7 minutes at room temperature, was included. Gating was based on FSC, SSC and positive immunoreactivity for the relevant proteins (i.e., α-Syn, Sj-1 and PIPKγ). A total of 2,000-4,000 gated cells were counted in each experiment unless indicated differently.

### Transferrin Endocytosis

Measurement of transferrin endocytosis were performed as previously described with some modifications. Cells were grown in 12-wells plates, on cover slides that were pre-treated with poly-D-lysine (100 μg/ml) for 1 hour. On the day of the experiment, cells were serum-starved for 1.5 hours, or treated with media lacking B27 supplementation (primary neurons). Cells were then conditioned in 25 μg/ml of 568-Tf (Molecular Probes, Invitrogen, Rhenium, Israel) in clear DMEM at 37° C. for the time indicated. When specified, induction of FRB-FKBP dimerization and recruitment of Inp45p to the PM was achieved with the addition of rapamycin (500 nM) in DMSO (0.5% v/v). After two washes with ice-cold PBS, cells were acid washed at pH 5.3 (0.2 M sodium acetate, 0.2 M sodium chloride) on ice for 1.5 min, to remove surface-bound transferrin. Cells were then washed 2 additional times with ice-cold PBS, fixed in 2% paraformaldehyde (PFA) for 20 min on ice and processed for ICC.

### PI4,5P₂ Detection by PH-PLCδ1-GFP Biosensor

HeLa cells were grown on cover slides coated with poly-D-Lysine, in 12-well plates. Cells were co-transfected with PH-PLCδ1-GFP and either WT, A30P, E46K, A53T, K10,12E or K21,23E α-Syn expressing plasmids, using JetPRIME transfection reagent polyplus (Tamar, Rehovot, Israel). Forty-eight (48) hours post transfection, cells were incubated with 50 μg/ml Alexa-647 Concanavalin (Con)A (molecular probes, Invitrogen, Rehovot, Israel) in DMEM, at 37° C. for 10 minutes; washed in serum-free DMEM; and fixed with 4% paraformaldehyde for 10 minutes, on ice. Cells were then washed one more time and permeabilized with 0.2% Triton X-100 in blocking solution (1.5% BSA in PBS) for 5 minutes at room temperature. Cells were incubated with anti α-Syn antibody, C20 (Santa Cruz, Dallas Tex., US) at 1:500 dilution, overnight at 4° C., followed by a secondary ab. Membrane to cytosolic signal ratio of PH-PLCδ1-GFP was calculated using the NIS-Element AR Analysis 4.20.02 64-bit software (Nikon, Agentek, Tel Aviv, Israel). Membranes were defined by the ring-shaped ConA signal around the cell and differentiated from the cytoplasm of the cells.

### Immunocytochemistry (ICC)

Primary neurons grown on cover slips, at 4 or 14 DIV were gently washed with warm HBSS (Biological Industries, Beit-Haemek, Israel), fixed with 2% PFA for 20 minutes at room temperature and permeabilized with 0.5% saponin in 1% (w/v) BSA for 30 minutes, at room temperature. Cover slips were then incubated for two hours at room temperature with the indicated primary antibodies (Table 2) in 1% (w/v) BSA, followed by 3 washes in PBS, 5 minutes each. Slides were then incubated with appropriate secondary ab, washed, and mounted in Vectashield® mounting medium (Vector-labs, Burlingame, Calif. USA).

### Immunohistochemistry for Mouse Brain Sections

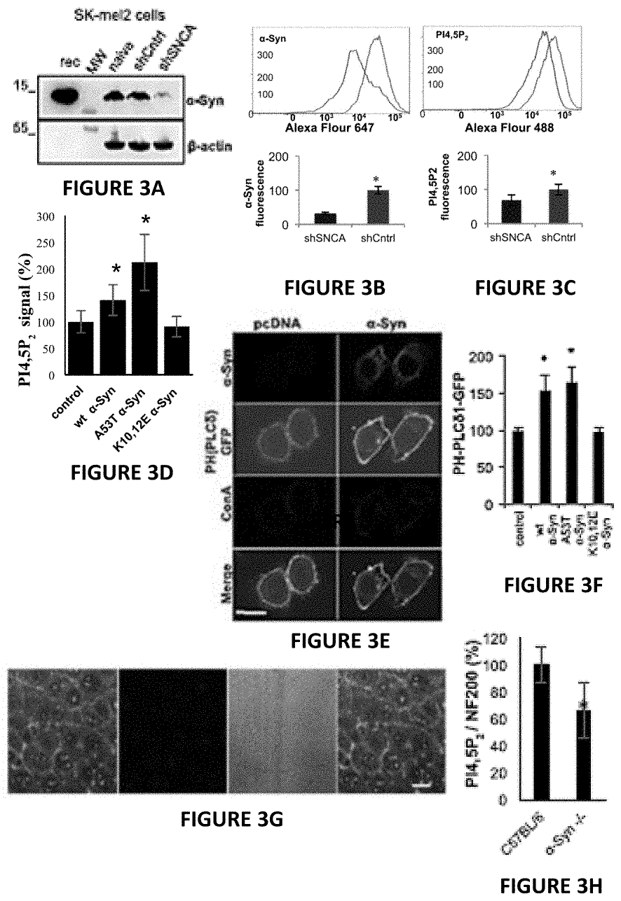

Paraffin-embedded, coronal mouse brain sections (6 μM) were processed for immunostaining as previously described. The antibodies used are listed in Table 2. Images were acquired using a Zeiss LSM 710 Axio Observer confocal Z1 laser scanning microscope, equipped with an argon laser 488, Diode 405-430 laser and HeNe 633 laser. Images at FIGS. 3G; 5A and 11B were captured at higher resolution using Nikon's A1R+ confocal microscope, equipped with an ultrahigh-speed resonant scanner and high-resolution digital galvano scanner, with four laser unit LU-N4S. Per each experiment, the exciting laser, intensity, background levels, photo multiplier tube (PMT) gain, contrast and electronic zoom were maintained constant. Antibody specific background was subtracted. The focus of each picture was obtained by choosing the plane with greatest fluorescent signal.

### Immunohistochemistry for Human Brains

Slides containing formalin fixed, paraffin embedded brain sections of advanced PD and controls, immunoreacted with anti SMI-32 ab or anti α-Syn antibody (BD Transduction Labs), were provided by the Multiple Sclerosis Society Tissue Bank. Otherwise, slides were processed for immunostaining as previously described. The antibodies used are listed in Table 2. Images were acquired using a Nikon Ti Eclipse motorized inverted microscope with DIC, phase epi-fluorescence optics and Perfect Focus System (PFS). Equipped with a Nikon DS-Fi1 color CCD camera and NIS-Elements image acquisition software. Fluorescence images were acquired using a Zeiss LSM 710 Axio Observer confocal Z1 laser scanning microscope (as above). All images were taken using the same settings, and on the same day. The specific signal inside WMTs was quantified per area and normalized to the nonspecific signal outside of WMTs. Quantification of SMI-32, vGlut1 and TH immunoreactivity in the caudate were performed based on 6-10 fields per brain at ×20 magnification (Image J). Fields were chosen randomly. Images were taken and analyzed blindly to tissue classifying information.

Quantifications were performed blinded to treatments. To reduce experimental error, comparisons were made within slides that were processed and analyzed in parallel. Image series were analyzed with Image Pro Plus 6.3 (Media Cybernetics, Bethesda, Md., USA) or Fiji (Image J). An average value was calculated for each animal, followed by calculation of the average for the group. Data presented in percent of control cells when including different immunostaining events. Quantitation of the signal localized specifically to neurites was performed with Image J as recently described. Determination of neurite and axon length, number, and length of collateral branches (longer than 15 μm) was done by tracing an axon including its collateral branches, starting from the cell body throughout, using the segmentation plugin for neurite tracer in Image J.

Synaptophysin-pHluorin (sypHy) Imaging

Neurons were infected at 5 DIV with AAV1/2 hSyn:sypHy and were imaged at 13 DIV. Coverslips were placed in a field stimulation chamber (Warner Scientific, Hamden, Conn., USA) in an extracellular solution composed of the following (in mM): 150 NaCl, 3 KCl, 20 glucose, 10 HEPES, 2 CaCl₂), 2 MgCl₂, pH adjusted to 7.35 with NaOH at 310 mOsm. The solution also contained glutamate receptor antagonists APV (50 μM) and DNQX (10 μM) to avoid recurrent network activity. Neurons were imaged at room temperature every 6 seconds on a Nikon TiE inverted microscope equipped with a Neo5.5 Andor sCMOS camera, using an EGFP filter set (Chroma, Bellows Falls, VT, USA). After acquiring 6 baseline images (F₀), neurons were stimulated by applying 300 bipolar pulses at 20 Hz, each of 1 ms duration and 10V/cm amplitude, through parallel platinum wires. At the completion of the experiment, the culture was exposed to saline in which 50 mM of the NaCl₂ was replaced with NH₄Cl, to expose the total pool of vesicles (Fₘₐₓ). The background-corrected fluorescence values recorded for each synapse were normalized either by the peak response during the stimulation train, or by the size of the total pool of vesicles, as indicated. The rate of endocytosis was assessed by exponential fitting of the time course of the decay in fluorescence from its peak upon the completion of stimulation back to baseline values. To exclusively image exocytosis, the inventors added 1 μM BafA to the extracellular solution. BafA blocks the vesicle proton pump, thus masking the endocytotic segment of SV cycle without affecting the kinetics of endocytosis. Quantification was performed with the NIS-Elements software (Nikon), by placing equal circular regions of interests (ROI)s on 30-50 synapses in each field and extracting the background-subtracted average fluorescence value of each ROI. A local background was obtained adjacently to each ROI.

### Statistics

Comparisons between two groups were performed by two-tailed t-test. Additional comparisons were performed by one-way ANOVA and Dunnett test for correction for multiple comparisons (Prism 7). Data presented as mean±SD or mean±SE, as indicated. Significant differences were considered with P<0.05.

### Example 1

### Higher Density of Thinner Axons in Striatal WMTs of α-Syn Tg Mouse Brains

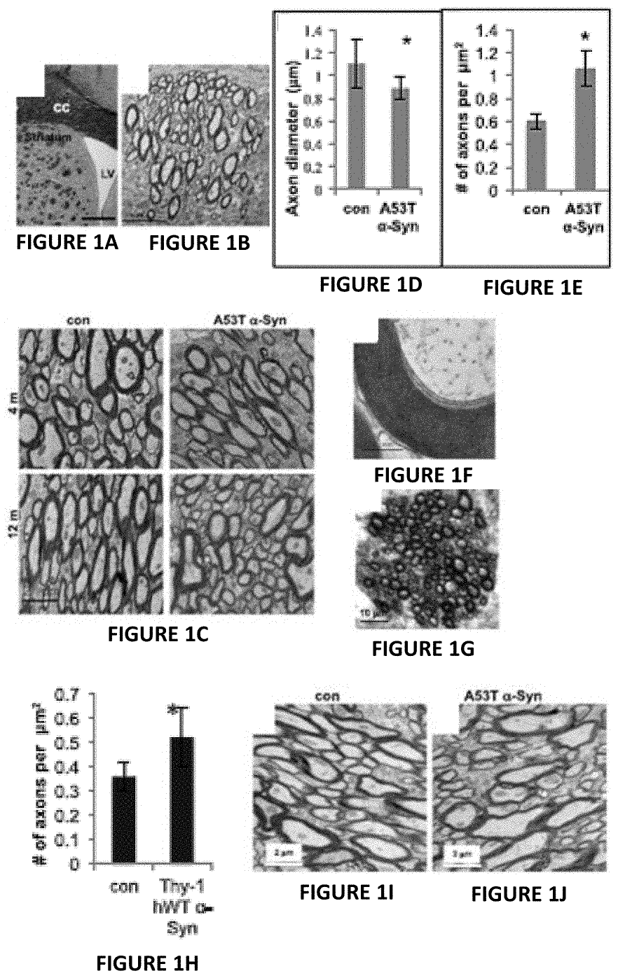

To investigate a potential role for α-Syn in axon integrity the inventors analyzed the ultrastructure of cross-sectioned axons, localized within striatal WMTs, in coronal sections of A53T α-Syn tg and control mouse brains. The inventors reasoned that the anatomical organization of the axons within the bundles and the relevance of the brain area to the disease could best fit the current focus of investigation. The tissue block was set to contain the dorsal striatum, just underneath the corpus callosum, using the size of the lateral ventricle as a reference for tissue position (FIGS. 1A-1B). Axon diameter and the density of axons were determined within WMTs of similar size (˜1.5-4×10⁻¹⁰ m²) and similar location at 2-4 months of age, representing healthy, fully myelinated mouse brains and at the age of 12-14 months, representing symptomatic mice (FIGS. 1C-1E). A significantly lower diameter was detected for axons of 12-14 months old A53T α-Syn tg (0.89±0.1 μm) than in age-matched control mouse brains (1.1±0.2 μm). Mean±SE of n=4 brains, 8-10 WMTs per brain; P<0.01, t-test. Surprisingly, the number of myelinated axons per μm² within WMTs was significantly higher in the A53T α-Syn tg (1.06±0.16) than in control brains (0.60±0.07). Mean±SE of n=4 brains, 8-10 WMTs per brain; P<0.05, t-test. In accordance with a recent report of the present inventors, no overt myelin ultrastructure or pathology was detected (FIG. 1F). That is, large axons, with a standard number and structure of lamellae were commonly detected in WMTs of the A53T α-Syn brains. However, compared with the control brains, a higher number of thinner axons, which are only sparsely myelinated were detected in the A53T α-Syn brain sections (FIG. 1C). Of note, differences in axon diameter or density between control and A53T α-Syn tg brains at young, 2-4 months of age were not statistically significant.

Similar results, indicating a higher density of axons within striatal WMTs, were detected in a second α-Syn tg mouse line, the Thy-1 hWT α-Syn mice. Semi-thick brain sections (1 μm) from 10-12 months old Thy-1 hWT α-Syn and control, age-matched non-transgenic littermates, were stained with methylene blue for visualization of myelin sheath (FIGS. 1G-1H). Comparing between WMTs of similar size and similar location (as above), the inventors determined 0.52±0.12 and 0.38±0.06 myelinated axons per μm² in Thy-1 hWT α-Syn and control mouse brains, respectively. Mean±SE of n=4 brains, 6-8 WMTs per brain. P<0.05, t-test.

The EM blocks containing coronal brain sections positioned at dorsal striatum, including the corpus callosum, were cut one more time in a position corresponding to sagittal brain sections (FIGS. 1I-1J). The sections, across the axons in corpus callosum of 12-14 months old A53T α-Syn and control mouse brains, revealed a high variability in axon diameter and axon density. No differences in the ultrastructure of axons were detected in the corpus callosum.

### Example 2

### Longer Axons and Collaterals in Primary Cultures of Mouse Neurons Expressing α-Syn

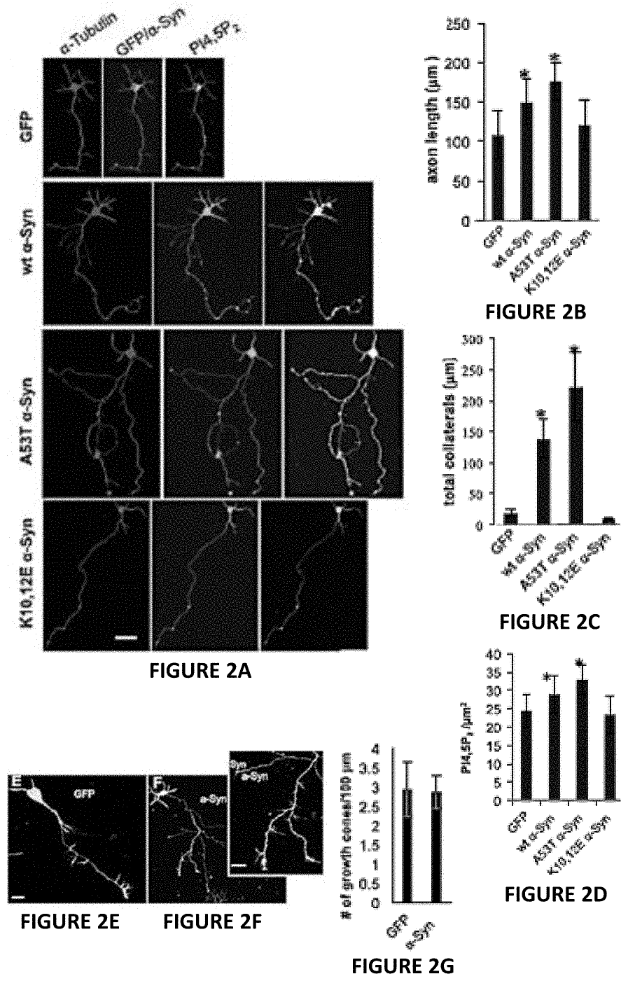

To investigate a potential involvement of α-Syn in elongation and/or branching of axons the inventors studied primary cultures of cortical neurons from α-Syn^{-/-} mouse brains. The cultures were transduced to express either human WT or A53T α-Syn, or a synthetic K10,12E α-Syn mutation generated by replacing two positively charged Lysine residues, within the KTKEGV repeat domain, with negatively charged Glutamic acid residues. In a previous study, this mutation was shown to interfere with α-Syn binding to membrane phospholipids. Control cells expressed a mock-GFP vector. Cells were fixed at 4 DIV and immunoreacted with antibodies against α-Syn, α-tubulin and the acidic phosphoinositide, PI4,5P2. The average length of the primary axon in control cortical neurons, transduced with the mock virus (in μm) was 108.50±29.9. Significantly longer axons were measured for WT α-Syn expressing (150.43±28.6) and furthermore for A53T α-Syn expressing neurons (175.97±23.8). Axon length in neurons expressing the K10,12E mutation (121.2±31.8) was not different than in control cells. Mean±SE; n>22 cells; P<0.05, one way ANOVA (FIGS. 2A-2B).

A dramatic effect on the total length of collaterals, extending from the main axon, was observed. The total length of collaterals (per axon) was ˜6.5-folds higher in WT α-Syn and ˜10 folds higher in A53T α-Syn than control neurons. In contrast, the length of collaterals in neurons expressing K10,12E α-Syn did not differ from control cells (FIG. 2C, mean±SE; n>22 cells; *P<0.05; one way ANOVA).

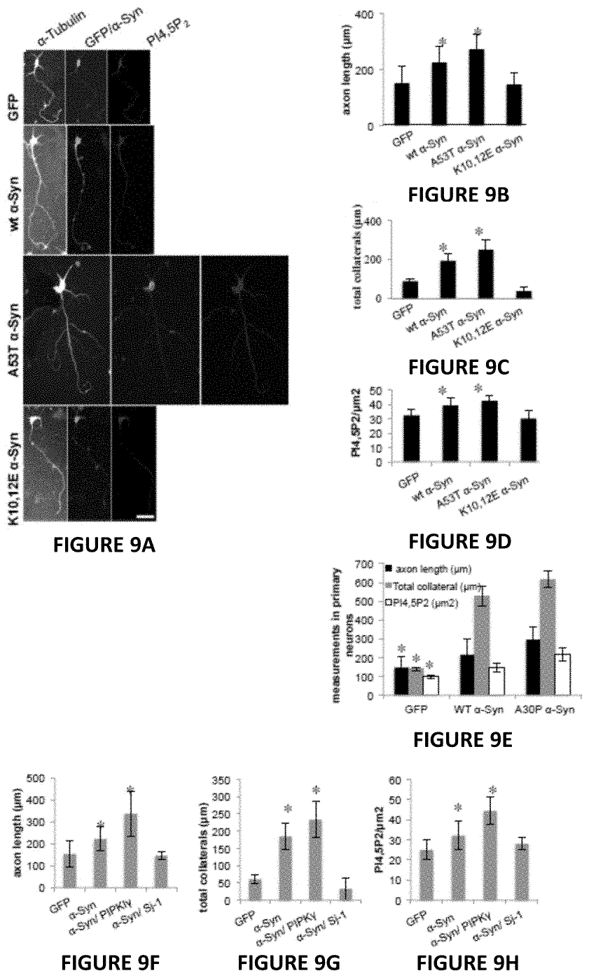

Importantly, similar to A53T α-Syn, expression of A30P α-Syn in primary mouse cortical neurons resulted in longer axons and longer collaterals compared with WT α-Syn expressing neurons (FIG. 9E). Moreover, α-Syn effects to enhance axon outgrowth were similarly detected in primary mouse hippocampal as well as mesencephalic neurons (FIG. 9).

### Example 3

### Altered PI4,5P₂ Levels in α-Syn Expressing Neurons

Phosphoinositides are a group of acidic phospholipids and PI4,5P₂ is implicated in axonal growth. Attempting to find out whether α-Syn associations with membrane phospholipids and its preference for acidic phospholipids may play a role in its effects to enhance axon outgrowth, the inventors co-immunoreacted the primary cortical neurons with anti PI4,5P₂ ab (FIG. 2A). PI4,5P₂ levels were determined per axon area (μm², FIG. 1D) by ICC. Setting PI4,5P₂ levels in control axons at 100%, the inventors detected significant ˜118% and ˜135% higher PI4,5P₂ levels in WT and A53T α-Syn expressing axons, respectively. The A30P mutation in α-Syn similarly increased PI4,5P₂ levels over WT α-Syn (FIG. 9E). However, expression of K10,12E α-Syn mutation had no detectable effects on PI4,5P₂ levels (n=22-24 cells, P<0.05, one-way ANOVA). α-Syn expression in hippocampal neurons resulted in similar increases in axonal PI4,5P₂ levels. That is, PI4,5P₂ levels in hippocampal axons (per μm²) expressing WT or A53T α-Syn were 122 and 131% higher (respectively) than in axons expressing a mock virus, set at 100% (FIG. 9D).

### Example 4

### α-Syn Expression does not Alter the Number of Growth Cones Per Axon

The increases in axon density demonstrated in FIG. 1 could potentially result from increases in the number of growth cones per μm axon. To assess growth cones, the inventors co-immunoreacted cultured cortical neurons at 4 DIV, expressing either mock-GFP or WT α-Syn, with an anti-GAP-43 antibody, a marker for growth cones and phalloidin, a marker for filamentous actin (FIG. 2E). A parallel immunoreaction included anti α-tubulin and phalloidin (FIG. 2F). Closely similar numbers of growth cones per μm axon were found for α-Syn expressing (2.85±1.0) and mock-GFP expressing cells (2.93±1.7) using the different antibodies (mean±SD, n=12-15 cells). Suggesting no effect for α-Syn expression on the number of growth cones per axon (FIG. 2G).

### Example 5

### α-Syn Regulates PI4,5P₂ Levels

In a series of experiments, the inventors assessed the associations of α-Syn with PI4,5P₂ and the specificity of these associations. Silencing endogenous α-Syn expression in SK-mel2 cells with shRNA resulted in ˜70% lower α-Syn levels relative to control cells, transduced with a scrambled shRNA (set at 100%; FIG. 3A). In accordance, PI4,5P₂ levels, determined by FACS were ˜34% lower in shSNCA compared with shCntrl expressing cells (FIGS. 3B-3C). Mean±SE; n=4,000 cells; P<0.05, t-test. PI4,5P₂ levels were next determined by FACS in HEK 293T cells, transfected to express α-Syn (FIG. 3D). A significantly higher PI4,5P₂ signal was detected in WT α-Syn (140.7±15.5%) and A53T α-Syn (211.9±33.2%) expressing cells than in control cells (set at 100%). Mean±SE, P<0.05, one way ANOVA. However, PI4,5P₂ levels in cells expressing the K10,12E α-Syn mutation were not different than control cells (FIG. 2D).

Similar results were obtained using a PH-PLCδ1-GFP molecular indicator of PI4,5P₂ levels. HeLa cells were transfected to express WT, A53T, K10,12E α-Syn or a mock vector, together with a plasmid expressing PH-PLCδ1-GFP. Cells were incubated with 647-concanavalin A (ConA) to mark the plasma membrane (FIGS. 3E-3F). The relative fluorescence intensity of PH-PLCδ1-GFP in plasma membrane to cytosol was set at 100% for the mock plasmid expressing cells. Significant 158.9±37% and 169.0±38% higher values were detected for WT and A53T α-Syn expressing cells, respectively. No effect for the K10,12E α-Syn mutation on PH-PLCδ1-GFP signal was detected. Mean±SE of 3 independent experiments; n=15-25 cells in each experiment, p<0.05, one-way ANOVA.

To find out if endogenous mouse α-Syn similarly enhances PI4,5P₂ levels in vivo, the inventors immunoreacted paraffin embedded, coronal brain sections from 2 months old α-Syn^{-/-} (C57BL/6JOlaHsd) and age-matched WT C57BL/6 mice with anti PI4,5P₂. The inventors found a significantly lower signal in α-Syn (61.8%) than control WT mice (set at 100%, FIG. 3G). P<0.01, t-test. N=5 mouse brains in each genotype. Importantly, PI4,5P₂ signal appeared in the nuclei and on the PM surrounding the cell body, supporting the specificity of the antibody-detected signal. The effect of mouse endogenous α-Syn on PI4,5P₂ levels was next tested in primary hippocampal neurons at 14 DIV prepared in parallel from WT C57BL/6 and α-Syn^{-/-} mouse brains (FIG. 3H). Cultured neurons were co-immunoreacted with anti PI4,5P₂ and anti-neurofilament (NF-200) antibodies. Normalizing the signal obtained for PI4,5P₂ to the signal detected for NF-200, the inventors detected a significant lower PI4,5P₂ signal in hippocampal neurons from α-Syn^{-/-} (˜66%) than C57BL/6 mouse brains (set at 100%). Mean±SD, n=5 fields, consisting of >10 cells; P<0.05, t-test.

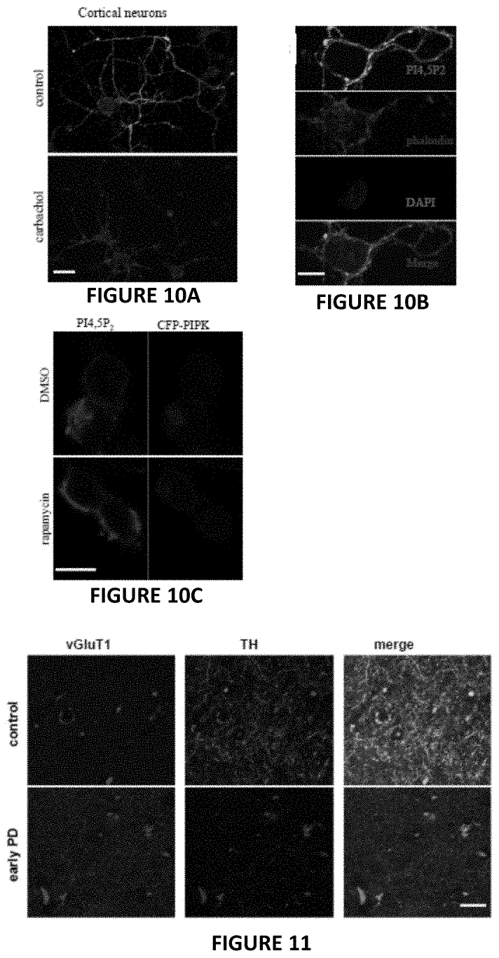

To confirm the specificity of the PI4,5P₂ signal that the inventors detected using anti PI4,5P₂ Ab in cortical neurons, PI4,5P₂ signal depletion following phospholipase C (PLC) activation was determined. Primary cortical neurons from α-Syn^{-/-} (C57BL/6JOlaHsd) mouse brains were treated with carbachol (1 mM at 5 DIV), a muscarinic agonist that activates PLC. Control cells were conditioned and treated in parallel but without the drug. Cells were fixed and immunoreacted with anti PI4,5P₂ ab (FIG. 10A). PI4,5P₂ signal in the carbachol treated neurons (25%) was dramatically lower than the signal detected in control neurons, treated with DMSO solvent (set at 100%; P<0.01, t-test). Importantly, in images captured at higher magnification, the loss of PI4,5P₂ signal is clearly detected on the PM of the cell body and throughout the axon, supporting specificity of the detected PI4,5P₂ signal (FIG. 10B). Similar results, showing loss of PI4,5P₂ signal following PLC activation in primary cortical neurons were obtained following activation of the muscarinic receptors with acetylcholine (10 μM) and pilocarpine (9.6 μM).

As an additional approach to study PI4,5P₂ signal specificity, the inventors expressed the rapamycin-induced translocatable CF-PIPK construct, which consists of active PIPKI-γ and CFP-FKBP. In HEK293T cells, CF-PIPK fluorescence was largely in the cytosol and moved to the PM with the addition of rapamycin. In accordance with the localization of CFP-PIPK, the detectable PI4,5P₂ signal was intracellular or on the PM, respectively (FIG. 10C).

### Example 6

### α-Syn Effect to Elongate the Main Axon and Collaterals Requires PI4,5P₂

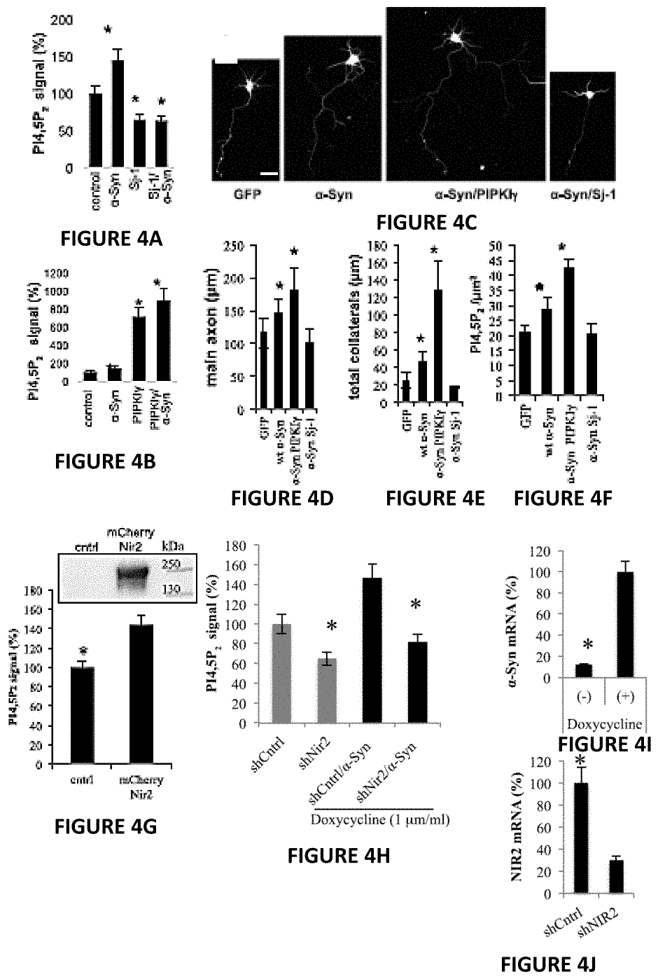

To investigate the potential involvement of PI4,5P₂ in α-Syn-dependent axonal outgrowth, the inventors tested the effects of synaptojanin-1 (Sj-1), a PI4,5P₂ 5-Phosphatase or PIPKIγ to alter PI4,5P₂ levels in HEK 293T cells expressing α-Syn (FIGS. 4A-4B). In cells co-expressing α-Syn and Sj-1 the increase in PI4,5P₂ levels, associated with its expression (e.g., ˜132% increase), was denied and PI4,5P₂ levels were below the levels of control cells, expressing the mock plasmid. In accordance, a dramatic ˜700% increase in PI4,5P₂ levels was detected with PIPKIγ expression and an additive effect of ˜20% was observed in cells co-expressing α-Syn and PIPKIγ (FIG. 4B). Mean±SD of n>3,000 cells; P<0.05, one-way ANOVA.

Next, the inventors co-expressed α-Syn either with Sj-1 or PIPKIγ in primary cortical neurons from α-Syn^{-/-} mouse brains by electroporation. Control neurons expressed WT α-Syn together with a mock plasmid or a GFP expressing vector. Cultures were fixed and immunoreacted with antibodies against α-Syn, tubulin and PI4,5P₂. The expression of Sj-1, PIPKIγ and GFP were visualized directly based on their GFP tag. Similar to the results above (FIG. 2), longer axons and longer collaterals were measured for α-Syn expressing cells (FIGS. 4C-4D). Specifically, the length of the main axon (in μm) was 116.5±22.1 in control cells and 145.7±21.7 in WT α-Syn expressing cells. Co-expression of WT α-Syn together with PIPKIγ further increased axon length (182.1±33.6) and co-expression of WT α-Syn together with Sj-1, eliminated the elongative effect of α-Syn (101.0±21.8) with a mean value that is lower than control cells expressing the mock-GFP plasmid (FIGS. 4D-4E). The total length of collaterals per axon in WT α-Syn expressing cells (47.1±6.7 μm) was longer than control cells (24.8±7.9 μm). Further increase in length of collaterals was observed with PIPKIγ (128.6±19.3 μm) yet, α-Syn effect to increase the length of collaterals was eliminated when co-expressed with Sj-1 (17.1±8.1 μm; FIG. 4F, mean±SD of n>20 cells, *P<0.05; one way ANOVA).

Quantifying PI4,5P₂ levels in the axons, the inventors found a significant increase with WT α-Syn expression (˜128%) and furthermore in cells expressing α-Syn and PIPKIγ (˜205%). However, PI4,5P₂ levels in axons expressing α-Syn and Sj-1 (˜98%) were not different than control cells (100%; FIG. 4F). Similar results, demonstrating the importance of PI4,5P₂ levels for α-Syn-mediated elongation of axons and collaterals were obtained in primary mesencephalic neurons (FIGS. 9E-9G). Based on these findings the inventors conclude that the mechanism through which α-Syn acts to elongate the axons is dependent on PI4,5P₂.

### Example 7

### The Regulatory Role of α-Syn on PI4,5P₂ Levels is Nir2-Dependent

To test the mechanism through which α-Syn increases cellular PI4,5P₂ levels, the inventors tested the potential involvement of Nir2-expression. HEK293T cells were transfected to express mCerry-NIR2 or a mock (control) plasmid. 72 hours post DNA transfection, PI4,5P₂ levels were determined by FACS. In line with a previous report, over-expressing mCerry-Nir2 in HEK 293T cells increased PI4,5P₂ levels (FIG. 4G). To find out whether Nir2 expression is required for α-Syn-mediated increases in PI4,5P₂ levels, the inventors utilized SH-SYSY cells, that inducibly express α-Syn in a Tet-On control. Cells were infected to silence NIR2 expression using a viral vector expressing shNIR2. Control cells were infected with shCntrl. Five days post infection, doxycycline (1 μM) was added to the cells to activate α-Syn expression. PI4,5P₂ levels were determined by FACS following 3 days of incubation with doxycycline. The efficacy of shNIR2 to silence Nir2 expression was tested by qPCR and the levels were found to be 70% lower than in cells infected with the scrambled shRNA (set at 100%, FIG. 4I). α-Syn levels of expression were ˜25 folds higher with doxycycline (FIG. 4J). The results show that doxycycline enhanced α-Syn expression resulted ˜146% higher PI4,5P₂ signal. However, the effect of doxycycline induced α-Syn-expression on PI4,5P₂ signal was abolished when cells were infected to silence Nir2 expression (FIG. 4H, mean±SD of n=3 different experiments; n=>3,000 cells in each treatment).

### Example 8

### Higher PI4,5P₂ Levels in Striatal WMTs of α-Syn Tg Mouse Lines

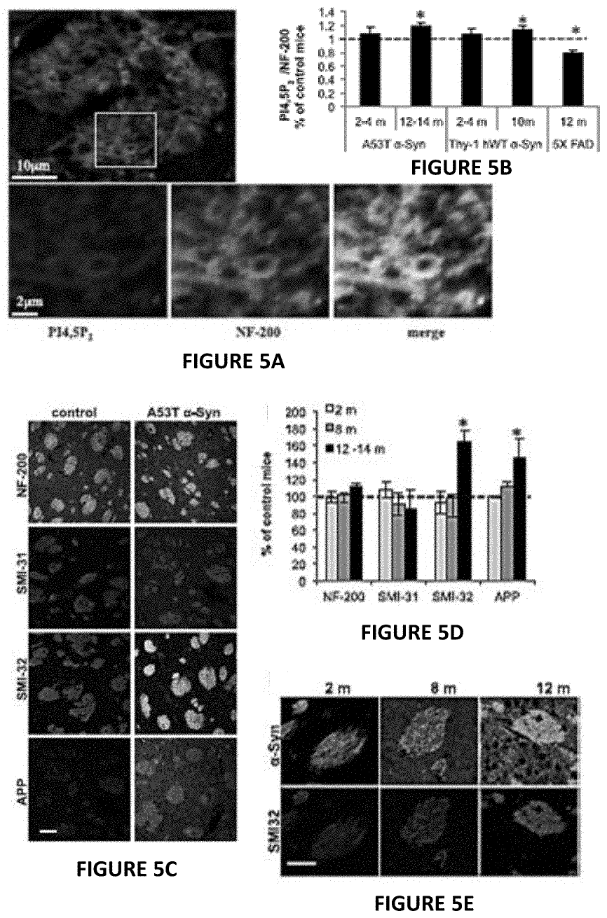

To draw a line between the findings in mouse brains, showing a higher density of axons within WMTs and the findings in primary neurons, showing an effect for α-Syn to enhance axon outgrowth, the inventors next determined PI4,5P₂ levels in striatal WMTs of α-Syn tg and control brains. Paraffin embedded brain sections of healthy young and symptomatic old mice of two mouse models, the A53T α-Syn and the Thy-1 hWT α-Syn mice were analyzed by immunohistochemistry (IHC). The respective age and genotype-matched control mice were analyzed in parallel (see methods). The position of the sections was set as above (FIG. 1A). Brain sections were co-immunoreacted with PI4,5P₂ and NF-200 antibodies (FIG. 5A). PI4,5P₂ levels were normalized to NF-200 signal, obtained within WMTs (per area). Setting PI4,5P₂ to NF-200 ratio of age and genotype-matched control mouse brains at 100%, the inventors detected ˜109% and significant ˜120% higher ratio in 2-4 and 12-14 months-old A53T α-Syn mouse brains, respectively. Similar results, showing ˜107% and significant ˜115% higher ratio were detected in WMTs of 2-4 and 10 months-old Thy-1 hWT α-Syn mouse brains, respectively (FIG. 5B; mean±SE of n=4 brains; P<0.05, one-way ANOVA). In a control experiment, PI4,5P₂ to NF-200 signal ratio was determined in WMTs of 12 months-old 5XFAD mice, modeling Alzheimer's disease. Importantly, in contrast to the PD mouse models, the results show a significant lower (˜70%) ratio in WMTs of old 5XFAD mice than in control brains (100%). Mean±SE of n=4 brains; P<0.05. Together, specific higher PI4,5P₂ levels per axon were detected in striatal WMTs of old, symptomatic, α-Syn tg mouse lines.

### Example 9

### Evidence for Axonal Injury within WMTs of α-Syn Tg Mouse Brains

To find out whether the effects of α-Syn to increase axon density within WMTs are associated with its toxicity, the inventors analyzed brain sections of A53T α-Syn at 2, 8 and 12-14 months, and age-matched control mice by IHC. Brain sections were immunoreacted with an anti SMI-32 antibody, which recognizes the non-phosphorylated epitopes on the neurofilament proteins and known for its immunoreactivity with corticostriatal axons. In addition, the sections were tested for SMI-31 immunoreactivity, which recognizes phosphorylated neurofilament. Consecutive brain sections were immunoreacted with an anti-amyloid precursor protein (APP) antibody, as a marker for axonal injury. The signal obtained within WMTs for each of these markers, in each of the tested age groups, was set at 100% for control mice. No differences were detected up to 8 months of age. In line with evidence for increases in axonal density within WMT (FIG. 1), the inventors detected a significant 165±18% higher SMI-32 immunoreactive signal in the α-Syn tg mouse brains. Interestingly, the significantly 144±17% higher APP signal, detected in WMTs of 12-14 months old A53T α-Syn mouse brains, indicates the occurrence of axonal damage (FIGS. 5C-5D; mean±SD of n=5 mouse brains). In accord with the occurrence of axonal damage, the inventors detected an age-dependent increase in α-Syn immunoreactivity, determined with syn303 antibody, within SMI-32-positive striatal WMTs and also in striatal matrix (FIG. 5E).

### Example 10

### Plasticity of Corticostriatal WMTs of Human Brains at Early Stages of PD

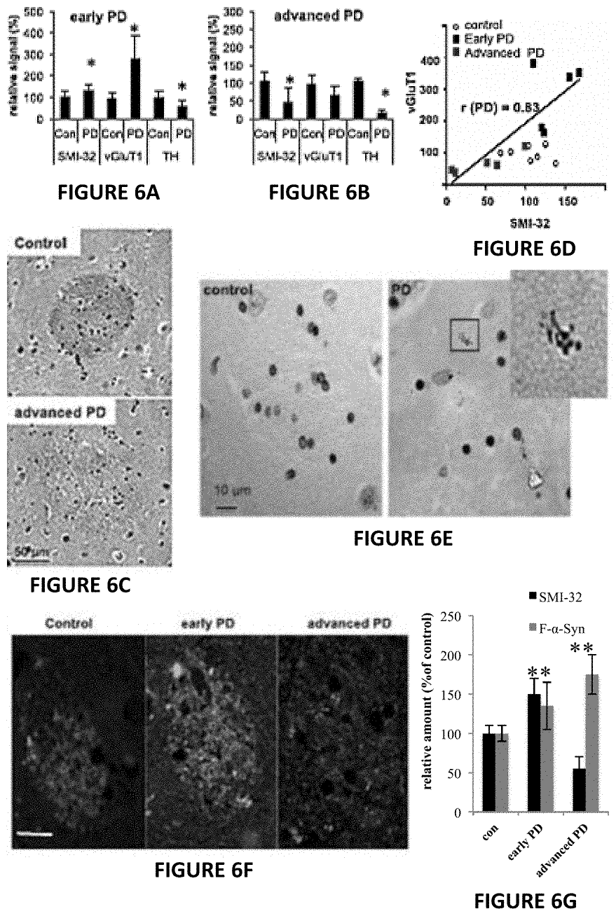

To study the corticostriatal glutamatergic connections in PD brains, the inventors determined SMI-32 signal in caudal WMTs and Vesicular glutamate transporter 1 (vGluT1) signal in corticostriatal terminals, at early PD (unified stages IIa and IIb) and advanced PD (Braak stages 5-6). The inventors reasoned that if glutamatergic plasticity indeed occurs in PD, then it is more likely to be detected at early rather than advanced disease stages. The results show a significant 135±24.8% higher SMI-32 signal in caudal WMTs of early PD cases (n=5) compared with control brains (n=7). Mean±SD; P=0.03, t-test (FIG. 6A). In contrast, a dramatic 46.9±38.1% lower SMI-32 signal was detected in advanced-PD cases (n=5) compared with control brains (n=7). Mean±SD; P=0.01, t-test (FIGS. 6B-6C). In accord, vGluT1 signal was considerably ˜283±87% higher in early PD (n=5, P=0.007, t-test) and 67.4±32.8% lower in advanced PD (n=5, P=0.05, t-test) compared with control brains (n=7; FIGS. 6A-6B and 11).

TH immunoreactivity was similarly determined in the caudate to test dopaminergic synapses in these human brains. TH signal was lower (61.9±8.6%) in early PD and considerably lower (16.5±9.7%) in the tested advanced PD cases (P<0.05, t-test; FIGS. 6A-6B and 11).

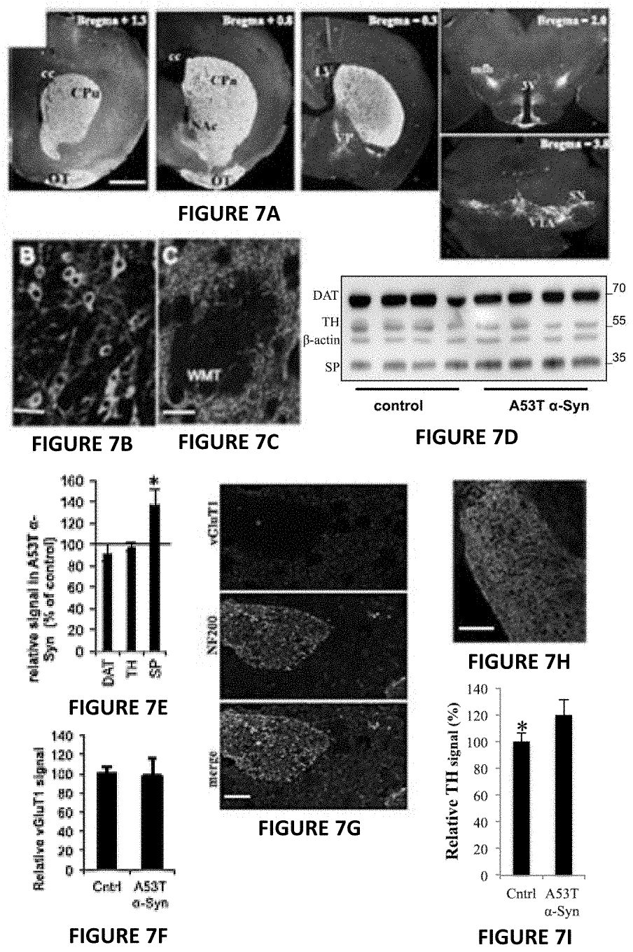

To find out whether SMI-32 immunoreactivity within WMTs correlates with vGluT1 immunoreactivity at corticostriatal terminals, the inventors co-immunoreacted PD and control brain sections with SMI-32 and vGluT1 antibodies. A strong positive correlation between SMI-32 and VGluT1 signals (Pearson's r value=0.83) was detected in the caudate of PD brains (n=10). However, no correlation could be detected for the control brains (Pearson's r value −0.21; n=7). The results therefore suggest that at early PD, increases in glutamatergic axons within WMTs correlate with increases in glutamatergic terminals in the caudal matrix. However, with progression of disease, both, glutamatergic axons and terminals are degenerated (FIG. 7D).

Together, the analysis in human brains at early stages of PD validate the occurrence of corticostriatal plasticity within WMTs, localized to the caudate and the findings indicating higher density of SMI-32-positive glutamatergic axons in WMTs of α-Syn tg mouse brains.

Evidence for α-Syn pathology was commonly detected within caudal WMTs of advanced PD brains. α-Syn pathology, detected in cross-sectioned axons within WMTs, consists mostly of Lewy neurites and observed as α-Syn-positive granulated signal. The densely hematoxylin-stained glia cells within WMTs were devoided of Lewy pathology (FIG. 6E). Importantly, similar to the finding in the α-Syn tg mouse brains (FIG. 5E) pathogenic forms of α-Syn protein, detected with an anti-filament α-Syn antibody were abundantly detected within WMTs and matrix early in PD, side by side with the increase in SMI-32 immunoreactivity (FIGS. 6F-6G). Further increases in pathogenic α-Syn were detected at advanced PD, however, these were accompanied with degeneration and loss of SMI-32 immunoreactive axons (FIGS. 6F-6G). Finally, the inventors assessed the diameter of axons within WMTs of early PD and control cases. A high degree of variability in axon diameter within a specific WMT (0.09-6.8 μm) was detected. The inventors reasoned that the high variability in axon diameter between WMTs within the same brain section denies meaningful comparisons between the groups.

### Example 11

### The Mouse Model Partly Recapitulates the Human Disease

The inventors further examined the degree in which the mouse model recapitulates alterations in dopaminergic and glutamatergic terminals, and the severity of disease. In line with the original description of this A53T α-Syn tg mouse line TH-immunoreactivity detected by IHC, appeared highly similar between 12-14 months old A53T and age-matched control mice (FIG. 7A). No differences in TH-immunoreactivity were detected in the SNc (FIG. 7B), WMTs or striatum (FIG. 7C).

The expression levels of TH, dopamine transporter (DAT) and synaptophysin, a marker for synaptic terminals, were next determined by a quantitative western blotting. Brain tissue punches containing the dorsal striatum of 12-14 months old mouse brains were homogenized to yield a total homogenate immediately after dissection. Protein samples of striatal homogenates (30 μg protein) were analyzed and normalized to actin levels detected on the same blot (FIGS. 7 D-7E). The western blot results confirmed the finding above, indicating no differences between the tested mouse genotypes for TH and DAT levels in the striatum. However, synaptophysin levels were significantly higher in striatal homogenates of A53T α-Syn (137%) than in control brains (set at 100%). Mean of n=5 brains in each genotype, P<0.05, t-test. Supporting increases in axons and synaptic terminals.

vGluT1 signal was quantified in the dorsal striatum, in paraffin-embedded, coronal brain sections of 12-14 months old A53T α-Syn tg and control mouse brains, by IHC. In addition, vGluT1 signal was assessed in tissue punches containing the dorsal striatum by western blotting. The results show no differences in vGluT1 levels between mouse genotypes (n=5-7 mouse brains in each genotype).

Interestingly, TH immunoreactive signal in the olfactory tubercle, which is innervated by dopaminergic neurons residing in the ventral tegmental area (VTA) and is part of the ventral striatum, was significantly 121±15% higher in the aged A53T α-Syn than in control mouse brains, set at 100% (FIGS. 6F-6G). Mean±SD of n=4 brains in each genotype, P<0.05, t-test.

Together, the results in murine brains do not show evidence for actual loss of dopaminergic axons or alterations associated with glutamatergic terminals. At the age 12-14 month, the transgenic mouse colony, which expresses the human mutant A53T α-Syn and backcrossed to the C57BL/6JOlaHsd α-Syn^{-/-} mouse genotype, recapitulates characteristic features of the human disease, including accumulation of α-Syn pathology and evidence for axonal injury, side by side with evidence for axonal growth, however, with no apparent loss of dopaminergic axons.

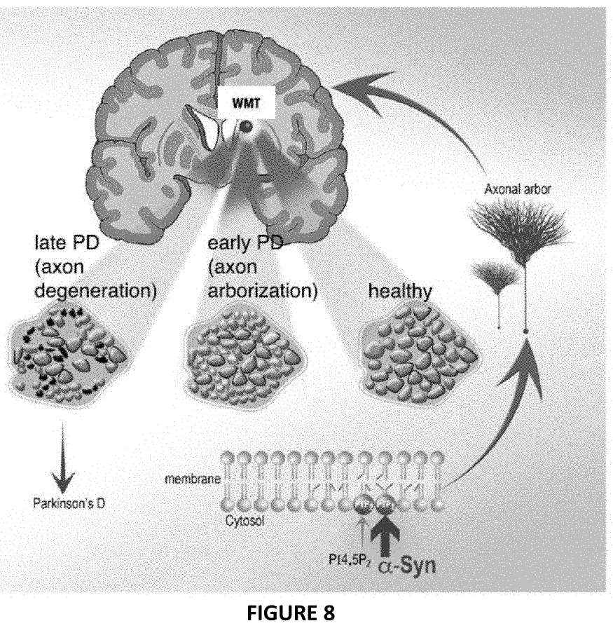

To this end, α-Syn is involved in the regulation of plasma membrane levels of PI4,5P₂ The increases in PI4,5P₂ levels enhance axon arbor. However, excessive growth of the axon is associated with accumulation of damage and is implicated in mechanisms of Parkinson's disease (FIG. 8).

### Example 12

### α-Syn Colocalizes with Phosphorylated AP2 (pAP2) and PIP2 on Clathrin Coated Pits (CCP)

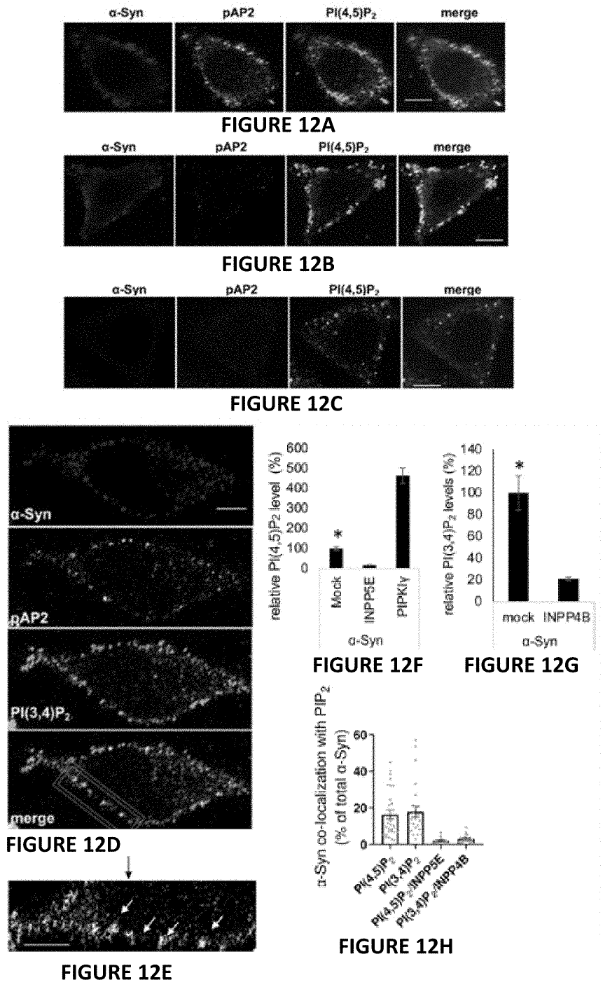

Phosphorylation at Thr156 of μ2 subunit of the clathrin adaptor AP2 starts following its binding to PI4,5P₂ at the initiation of a CCP and throughout vesicle lifetime. The inventors analyzed SK-Mel2 cells, which express detectable levels of endogenous α-Syn protein. The immunoreactive signal for pAP2, observed by ICC, appeared on the plasma membrane of the cells. This signal colocalized with the signal obtained for α-Syn, using anti α-Syn antibody (ab21976, FIG. 12A). To assess the specificity of pAP2 signal the inventors utilized a specific inhibitor (LP-935509) for Numb-associated kinases (NAKs), that phosphorylate the μ2 subunit of AP2 (42-45). pAP2 signal was dramatically reduced in cells treated with the LP-935509 inhibitor (10 for 3 hours) and no obvious colocalization of α-Syn and pAP2 could be detected (FIG. 12B). The specificity of α-Syn signal was confirmed in cells that their α-Syn expression was silenced with shSNCA and treated with the LP-935509 inhibitor. The results show a substantial loss of both signals, α-Syn and pAP2 (FIG. 12C).

To confirm that α-Syn colocalizes with pAP2 on CCP, the slides were immunoreacted also with antibodies against PI4,5P2 (FIGS. 12A-12C) or PI3,4P2 (FIG. 12D). The results show a high degree of colocalization for the immunoreactive signals obtained for α-Syn, pAP2 and either PI4,5P2 or PI3,4P2 (FIGS. 12A-12E).

The specificity of PI4,5P2 and PI3,4P2 signals were assessed in control HEK293T cells, transfected to express either the inositol polyphosphate-5-phosphatase E (INPP5E), that dephosphorylate the 5-phosphate of PI4,5P2; or the inositol polyphosphate-4-phosphatase B (INPP4B), that dephosphorylate the 4-phosphate of PI3,4P2; or type Iγ PI4P-5-kinase (PIPKIγ), that produces PI4,5P2. Importantly, PI4,5P2 signal was dramatically lower in cells expressing the INPP5E phosphatase, and ˜4.5 fold higher with the expression of PIPKIγ (FIG. 12F). In accord, PI3,4P2 signal was dramatically lower with the expression of INPP4B phosphatase (FIG. 12G).

Using a program-based method, the inventors scanned the ICC images obtained for the SK-MEL2 cells to identify positive pixels in each channel and the colocalizing pixels within the channels. A portion of α-Syn signal specifically colocalized with PI4,5P2 (˜16%) and PI3,4P2 (˜18%). Colocalization of α-Syn with PI4,5P2 or PI3,4P2 was diminished following the expression of INPP5E or INPP4B, respectively, confirming the specificity of the results (FIG. 12H).

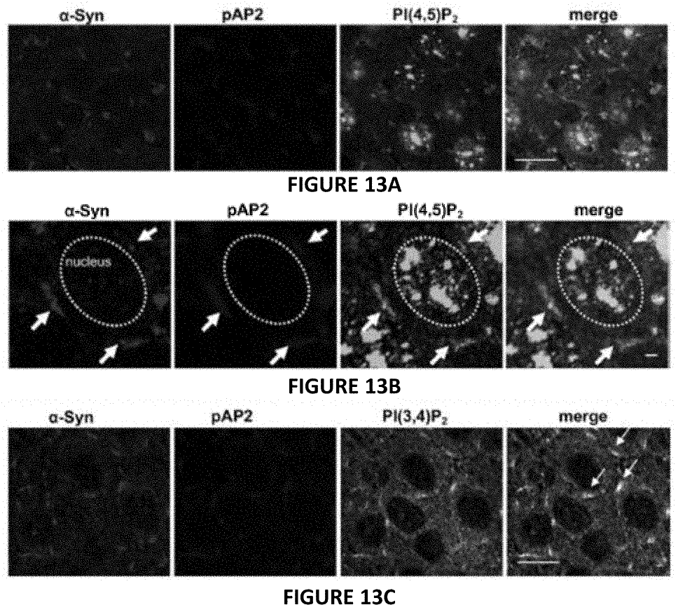

The inventors next validated the results indicating colocalization for the immunoreactive signals obtained for α-Syn, pAP2 and PI4,5P2 in coronal brain sections from an A53T α-Syn tg mouse by immunohistochemistry. The results show a strong nuclear signal with the PI4,5P2 antibody. In addition, colocalization between α-Syn, pAP2 and PI4,5P2 is detected surrounding the cells in the hippocampus (FIGS. 13A-13B), as well as additional brain regions. Similar results were obtained in a similar set-up, were PI3,4P2 ab was replaced with PI4,5P2 ab (FIG. 13C). Although nuclear PI3,4P2 signal is substantially weaker.

These results suggest that endogenous α-Syn localizes, at least in part, to PI4,5P2/PI3,4P2-positive endocytic clathrin-coated pits, consistent with a possible function in CME.

### Example 13

### α-Syn Involvement in Endocytosis of Transferrin Associates with Alterations in Cellular PIP₂ Levels

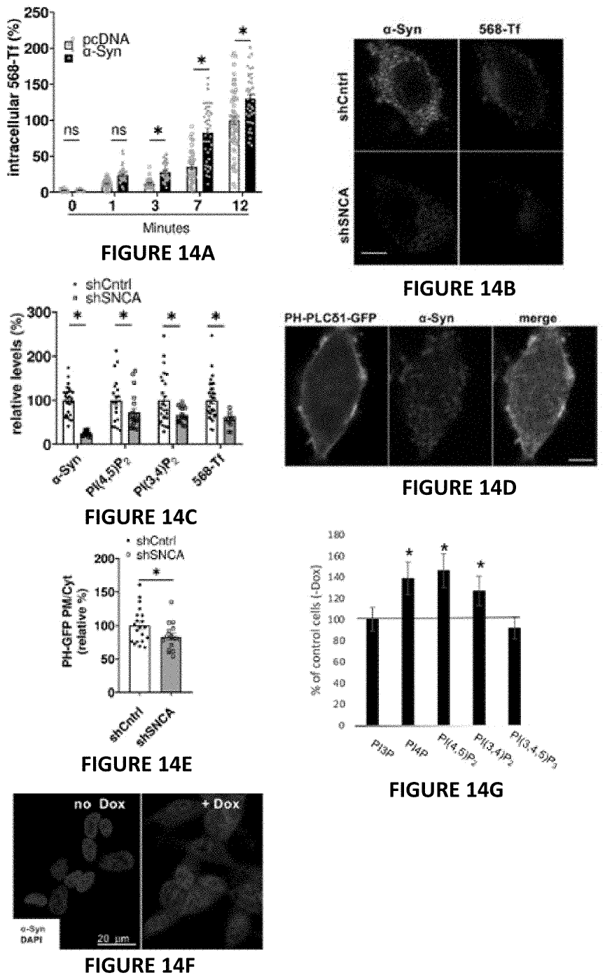

Endocytosis of fluorescently labelled transferrin (568-Tf) was utilized as a functional readout for CME. The kinetics of 568-Tf endocytosis was measured in HEK293T cells, transfected to express α-Syn or a mock pcDNA plasmid. Seventy-two hours post DNA-transfection cells were conditioned in serum free DMEM to enhance the localization of transferrin receptor at the plasma membrane. 568-Tf was applied for 0-12 minutes at 37° C. to allow binding and internalization of 568-Tf. Cells were then acid-washed to remove surface-bound 568-Tf and processed to visualize and quantify 568-Tf by confocal microscopy (FIG. 14A). A higher degree of 568-Tf endocytosis was detected in α-Syn over-expressing cells compared with the mock expressing cells. The significant differences were observed starting from 3 minutes of incubation and furthermore at 7 and 12 minutes (FIG. 14A).

In complement experiment, endogenous α-Syn expression was downregulated in the SK-Mel2 cells using shSNCA and were 24% of the levels detected in control cells, infected with shCntrl (FIGS. 12C and 14B-14C). α-Syn levels were kept down regulated for at least 14 days and experiments were performed during this time window. Endocytosis of 568-Tf, following 7 minutes of incubation, was significantly (55%) lower in shSNCA than in shCntrl cells (set at 100%, FIGS. 14B-14C). In agreement with a recent report by the present inventors, silencing α-Syn expression resulted in significantly lower levels of PI4,5P₂ (73%) compared with control cells (set at 100%). Similarly, PI3,4P₂ levels were also lower (66%) with silencing α-Syn expression (ICC).

To verify that the observed loss of PI4,5P₂ was specific to the plasma membrane, the inventors utilized the PH(PLCδ)-GFP biosensor for PI4,5P₂ detection. SK-Mel2 cells expressing either shSNCA or shCntrl were transfected to express PH(PLCδ)-GFP. The signal ratio of GFP fluorescence in the plasma membrane to cytosolic GFP was calculated and used to indicate plasma membrane PI4,5P₂. Importantly, the results obtained with the PH(PLCδ)-GFP biosensor were highly similar to the results with anti-PI4,5P₂ antibody (FIGS. 14D-14E) and confirmed the significant reduction in plasma membrane PI4,5P₂ in α-Syn-depleted cells (i.e., 83% of PH(PLCδ)-GFP signal ratio compared to control cells, set at 100%).

To assess the general effects of α-Syn on PIPs, the inventors utilized an inducible SH-SY5Y cell line, expressing α-Syn under the control of Dox (FIGS. 14F-14G). α-Syn expression was induced for 72 hours, and cells were processed for the detection of PIPs by FACS. Control cells that express a mock plasmid were treated in parallel. Significantly higher levels of PI4P, PI3,4P₂ and PI4,5P₂ were detected with inducing the expression of α-Syn compared with the control cells (set at 100%). In contrast, PI3P and PI3,4,5P₃ levels were not altered upon α-Syn overexpression (FIG. 14G).

These data suggest that α-Syn regulates the levels of PI3,4P₂ and PI4,5P₂ phosphoinositides that control CME of transferrin. The inventors therefore decided to test the hypothesis that α-Syn increases PIP2 levels to enhance CME.

### Example 14

### α-Syn-Mediated Endocytosis of Transferrin is PI4,5P₂-Dependent

To experimentally regulate the levels of PI4,5P₂, the inventors utilized an inducible enzymatic system to acutely deplete PI4,5P₂ from the plasma membrane. This system enables rapamycin-induced targeting of Inp45p, a PI4P-5-phosphatase, to the plasma membrane. HEK293T cells were transfected to co-express the inducible phosphatase together either with WT α-Syn or pcDNA mock plasmid. 48 hours post DNA-transfection, cells were processed simultaneously for 568-Tf endocytosis together with activation of the phosphatase with rapamycin (see methods).

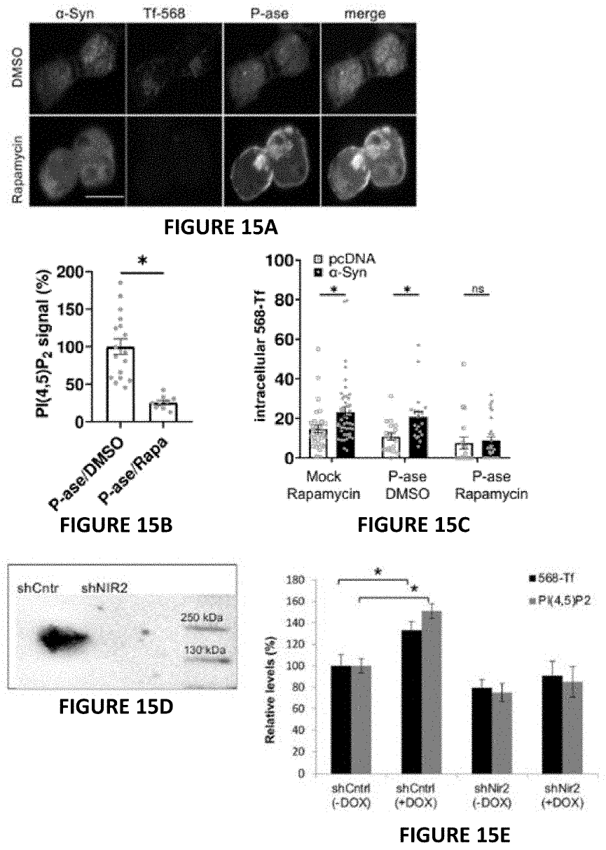

The Inp45p phosphatase is recruited to the plasma membrane in cells treated with rapamycin but remains in the cytoplasm in DMSO-treated cells (FIG. 15A). In accord, PI4,5P₂ levels were lower in rapamycin (26%) compared with the DMSO treated cells (set at 100%), demonstrating phosphatase activity (ICC; FIG. 15B). To find out whether plasma membrane PI4,5P₂ levels play a role in α-Syn′ effect to enhance endocytosis, the inventors quantified 568-Tf internalization in cells that co-express Inp45p together with WT α-Syn and treated with rapamycin or DMSO (FIGS. 15A and 15C). The results show that rapamycin-induced depletion of PI4,5P₂ completely abolished the ability of overexpressed α-Syn to stimulate CME (FIG. 15C), whereas CME was stimulated by α-Syn expression in cells treated with DMSO (vehicle), measuring a higher degree of 568-Tf internalization (193%) compared with the control cells that express a mock pcDNA (set at 100%). In control cultures, in which cells were transfected and treated in parallel but without Inp45p expression, the inventors found no effect for rapamycin on α-Syn-induced CME (FIG. 15C).

To confirm a role of PI4,5P₂ in α-Syn-mediated endocytosis of Tf-568, the inventors tested the significance of silencing Nir2 expression. An important function of Nir2 protein is the exchange of endoplasmic reticulum phosphatidylinositol (PI) with PM phosphatidic acid (PA), which is required for maintaining PM levels of PI4,5P₂. Nir2 expression was silenced with shNir2 in the inducible α-Syn expressing SH-SY5Y cell line, resulting in ˜70% lower Nir2 mRNA levels and lower protein levels of the respective levels detected in control cells, infected to express shCntrl (FIG. 15D). α-Syn expression was then induced with doxycycline for 72 hours and cells were analyzed by FACS (n>2,000 cells) to detect PI4,5P₂ and α-Syn levels. Sister cultures were analyzed by ICC (n=20-25 cells) to detect internalized 568-Tf (FIG. 15E). Inducing α-Syn expression with doxycycline resulted in significantly higher PI4,5P₂ (151%) levels and in accord, higher internalization of 568-Tf (133%), compared with cells that were not treated to induce the expression of α-Syn (100%). However, in cells that their Nir2 expression was silenced, induction of α-Syn expression had no effect on PI4,5P₂ levels, nor endocytosis of 568-Tf. The results therefore suggest that interference with the homeostasis of PI4,5P₂ on the PM inhibited the effects of α-Syn to enhance endocytosis of transferrin.

### Example 15

### α-Syn Mutations Correlate Endocytosis of Transferrin with Changes in Plasma Membrane Levels of PI4,5P₂

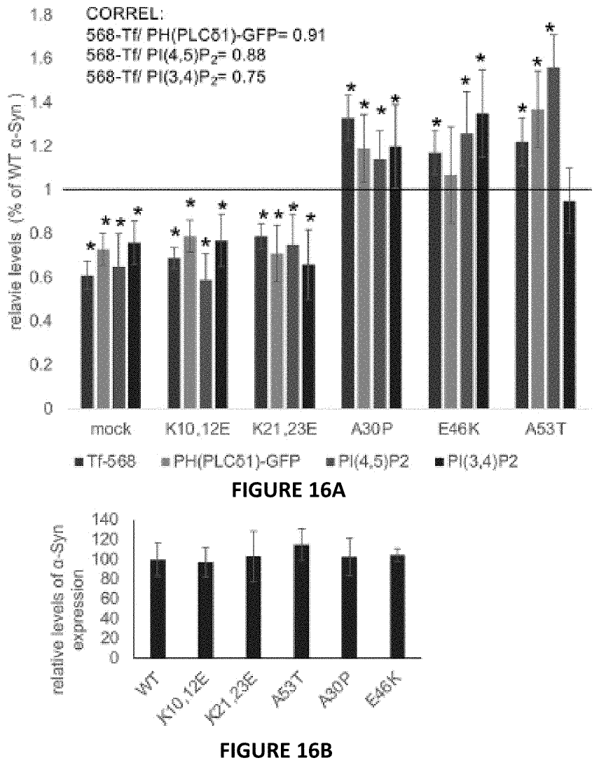

Endocytosis of 568-Tf and PIP2 levels were determined in HEK 293T cells, expressing either one of the following α-Syn forms, WT α-Syn; the PD-associated mutations in α-Syn, A30P, E46K or A53T; the synthetic K10,12E or K21,23E mutations. The synthetic mutations in α-Syn were generated by replacing two positively charged Lysine residues within the KTKEGV repeat domain, with negatively charged Glutamic acid residues. In a previous study, these K to E mutations were shown to interfere with α-Syn binding to membrane phospholipids. α-Syn expression, PI3,4P₂ and PI4,5P₂ levels were determined by FACS, using specific abs (n<2,000 cells; FIGS. 16A-16B) and plasma membrane levels of PI4,5P₂ were determined by the PH(PLCδ)-GFP signal ratio (as above; n=20-25 cells; FIG. 16A). The results show significantly higher levels of 568-Tf endocytosis, PI3,4P₂ and PI4,5P₂ in WT α-Syn than in the mock-plasmid expressing cells. Further increases over WT α-Syn were generally detected for these measurables with the PD-associated mutations, with the exception of the A53T effect on PI3,4P₂ and the E46K effect on plasma membrane PI4,5P₂ levels. The levels determined in the K-to-E mutations in α-Syn were lower compared with WT α-Syn-expressing cells (FIG. 16A). Comparable levels of α-Syn expression were detected for the tested α-Syn constructs (FIG. 16B).

A strong correlation between α-Syn effects on 568-Tf endocytosis and plasma membrane levels of PI4,5P₂ was noted (FIG. 16A; correlation coefficient [r]=0.91). Similarly, 568-Tf endocytosis correlated with total signal of PI4,5P₂ (r=0.88). 568-Tf endocytosis correlated also with PI3,4P₂ levels (r=0.75).

The inventors thus concluded that α-Syn increases PIP2 levels to facilitate CME and decided to test the hypothesis that it similarly acts to enhance SV endocytosis.

### Example 16

### α-Syn Accelerates the Rate of SV Endocytosis Alongside with Reducing the Fraction of Released SVs

The involvement of α-Syn in SVs cycling was tested using Synaptophysin-2XpHluorin (sypHy) (52,53). The pH-dependent fluorescence of sypHy, which is quenched in intact acidified SVs, increases upon exocytosis. After endocytosis and re-acidification of the SV lumen, fluorescence is re-quenched and returns to baseline. Primary hippocampal neurons prepared from α-Syn^{-/-} (C57BL/6JOlaHsd) mouse brains were infected to express sypHy together with one of the following α-Syn forms, WT α-Syn, the A30P, E46K, A53T mutants, or the two K to E mutations. mCherry served as a control for infection efficacy. SV cycling was measured at 13 days in vitro (DIV) by imaging sypHy before, during and after the delivery of 300 stimuli at 20 Hz (13,54). NH₄Cl saline was applied following the return of fluorescence to baseline, to alkalinize all intracellular compartments, thus exposing the total size of the SV pool (Fₘₐₓ).

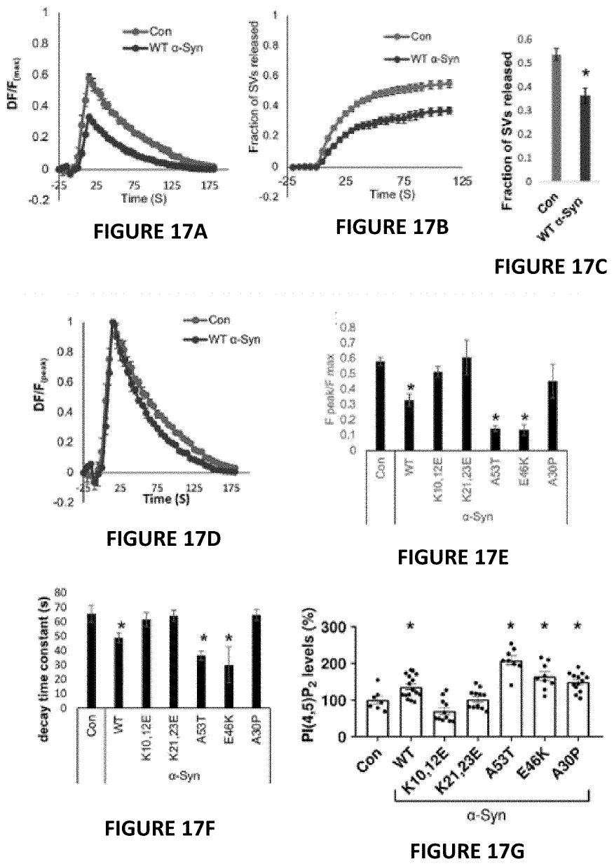

The results show that WT α-Syn expression over the α-Syn^{-/-} background inhibits the extent of SV cycling, represented by a lower peak fluorescence (Fₚₑₐₖ/Fₘₐₓ); FIG. 17A; n=50 synapses per image, 3 experiments. The lower peak level of the sypHy signal is in agreement with previous reports on an inhibitory role for α-Syn in exocytosis. A lower sypHy signal may arise either from a reduction in the number of SVs available for release, an acceleration of endocytosis or both. To assess specifically SV exocytosis, the inventors added bafilomycin-A (BafA) to the bath. BafA inhibits re-acidification of the SVs after endocytosis and thus, sypHy measurements performed in its presence report exclusively exocytosis. Indeed, in the presence of BafA, the cumulative sypHy signal in WT α-Syn expressing neurons was lower than in control neurons (FIGS. 17B-17C), indicating a reduction in the total secretory capacity of the presynaptic terminals, as has been previously reported. Importantly, normalizing the traces by the peak fluorescence obtained at the completion of stimulation (ΔF/Fₚₑₐₖ) revealed that the kinetics of the decay of sypHy was accelerated by the expression of WT α-Syn (FIGS. 17D-17F). Thus, in addition to its inhibitory effect on the exocytotic segment of the SV cycle, α-Syn also accelerates the rate of endocytosis.

The PD-associated mutations in α-Syn, E46K and A53T, further inhibited SV recycling (FIG. 17E) and in accord, further accelerated the rate of endocytosis (FIG. 17F). However, the A30P mutation and both K to E mutations were not different from control cells in their effects on SV recycling (FIGS. 17E-17F). Together, measurements of α-Syn effects on SVs cycling, as determined by sypHy, reveal its complex effects on SV pools and architecture. However, considering the actual segment of SVs trafficking, α-Syn appears to accelerate the rate of endocytosis.

The inventors next assessed PI4,5P₂ levels in primary neurons infected to express WT α-Syn or the specified mutations as above. At 13 DIV, neurons were fixed and processed for ICC with anti α-Syn and anti PI4,5P₂ antibodies. Similar to the results in HEK 293T cells (FIG. 16), the inventors found that expression of α-Syn mutations in hippocampal neurons differentially affected PI4,5P₂ levels (FIG. 17G). That is, WT α-Syn increased PI4,5P₂ levels (136%) over the levels detected in control cells (set at 100%); the PD-associated A30P, E46K and A53T mutations further increased PI4,5P₂ levels (150-208%), however, PI4,5P₂ levels in primary neurons expressing the K to E mutations did not differ from control cells.

The results further demonstrate a correlation between α-Syn-dependent increases in PI4,5P₂ levels and its capacity to enhance the rate of endocytosis. That is, an inverse correlation of r=−0.75 was calculated between the decay-constant of sypHy signal and PI4,5P₂ levels with the different α-Syn mutations. Excluding A30P mutation, which appears ineffective in SV endocytosis, yet increases PI4,5P₂ levels, results in a stronger correlation (r=−0.87).

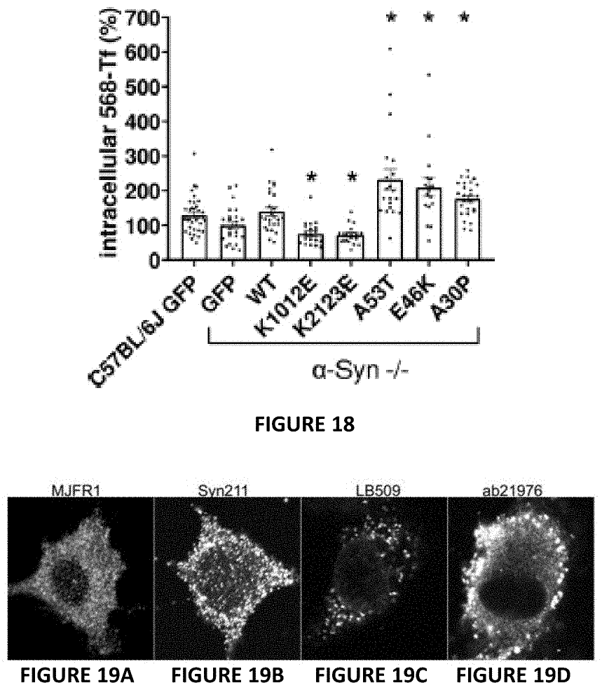

To further assess the effects of A30P mutation in CME, in neuronal cells, the inventors next determined 568-TF endocytosis in primary cortical neurons prepared from WT (C57BL/6J) or α-Syn^{-/-} (C57BL/6JOlaHsd) mouse brains. α-Syn^{-/-} neurons were infected to express either WT α-Syn, the specified mutations as above or a mock GFP plasmid. Neurons obtained from the WT mouse brains were also infected to express the GFP plasmid. At 8 DIV, neurons were conditioned with B27-free medium for 90 minutes, 568-Tf was added to the cells for 7 minutes at 37° C., cells were then acid-washed, to remove surface bound 568-Tf and fixed to determine direct fluorescence by confocal microscopy (FIG. 18). The result show highly similar levels of 568-Tf endocytosis in cortical neurons from C57BL/6J brains and α-Syn^{-/-} neurons infected to express WT α-Syn. Suggesting that the expression levels of α-Syn in the rescued α-Syn neurons and the WT C57BL/6J neurons are closely similar. WT α-Syn expression enhanced endocytosis (139%) over the levels detected in control α-Syn^{-/-} neurons expressing the mock GFP plasmid (set at 100%); further increases in endocytosis were detected for the PD-associated A30P, E46K and A53T mutations (177, 210 and 233%, respectively). Whereas the K to E mutations in α-Syn abolished the enhancing effects on endocytosis. All mutant α-Syn tested differed significantly from WT α-Syn in their effects on 568-Tf endocytosis. Thus, A30P mutation appears to effectively enhance endocytosis of 568-Tf and CME (FIG. 18). Yet, it interferes with α-Syn effects to enhance SV endocytosis at the synapse (FIG. 17).

### Example 17

### PI4P and PI4,5P2 Levels in Blood Cells Extract are Predictive of Parkinson's Disease

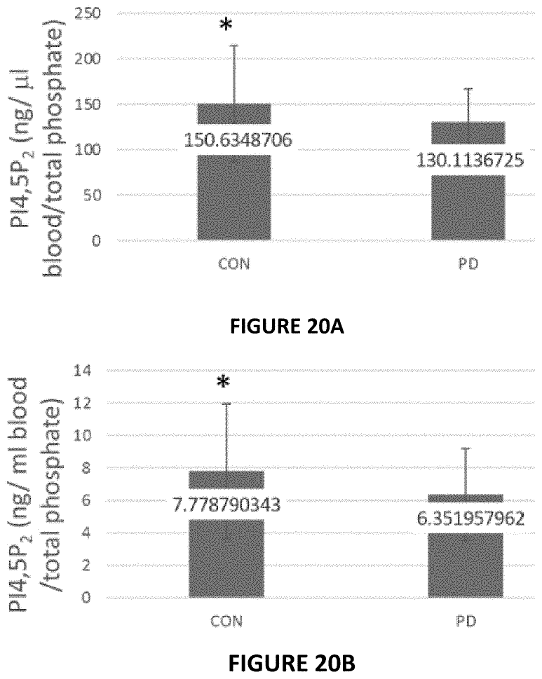

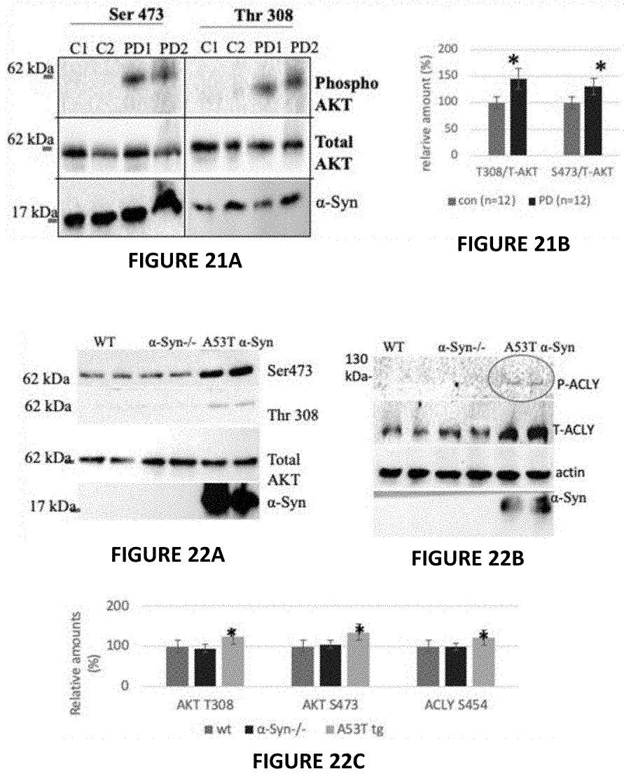

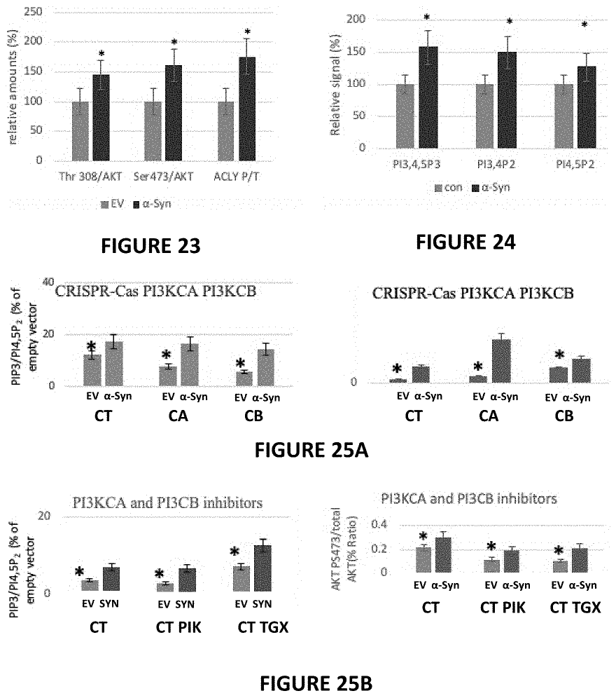

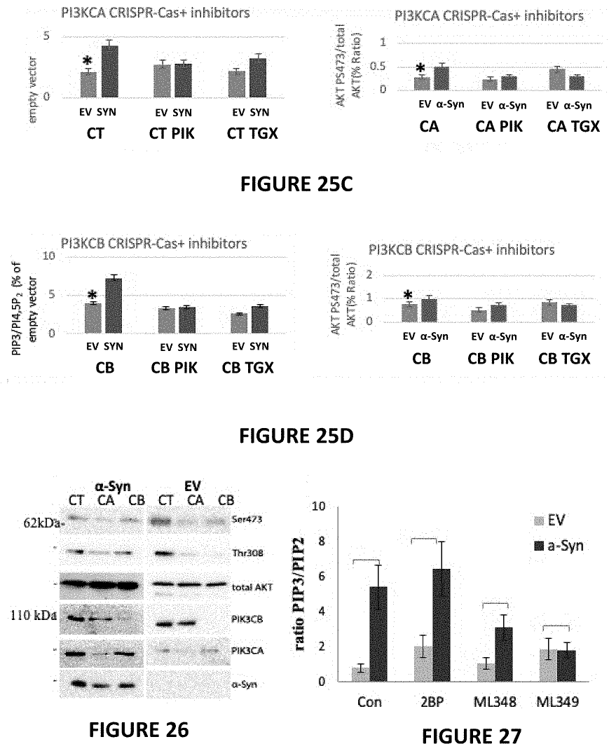

PI4P and PI4,5P2 were detected in lipid extractions from blood cells from human blood donors. Detection was showing with a reasonable assay linearity. No detection was observed for PI3P or PI3,4P2, which were below assay sensitivity limits (FIGS. 20A-20B).

The inventors have further extracted blood cells from samples of control (n=49) and PD (n=49) subjects. Polar lipids were extracted using a modified Folch extraction method. PI4,5P2 levels were determined by ELISA using anti-PI4,5P2 antibody (Echelon). The detected levels were normalized to the total amount of phosphates in the sample, which was determined by Malachite green.

The results show that PI4,5P2 levels were significantly lower in PD subjected compared to control (FIGS. 20A-20B). Accordingly, the inventors conclude that determining levels of phosphoinositide, such as PI4,5P2, can be used to determine or diagnose PD in a subject.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A method for treating a subject afflicted with any one of: an alpha-synuclein related disease, PD, and any combination thereof, comprising the steps:
a. determining whether at least one phosphoinositide is present in an amount: (i) decreased below a baseline; or (ii) increased above a baseline, in a sample obtained or derived from said subject; and
b. administering to the subject determined as having decreased or increased amount of said at least one phosphoinositide below or above said baseline, respectively, a therapeutically effective amount of pharmaceutical composition comprising any one of: an agent suitable for inhibiting or reducing alpha synuclein activity, pathogenicity, or both, an agent suitable for anti PD therapy, and any combination thereof,
thereby treating the subject afflicted with any one of: an alpha-synuclein related disease, PD, and any combination thereof.

2. The method of claim 1, further comprising a monitoring step (c) comprising at least once determining the amount of said at least one phosphoinositide in sample obtained or derived from said administered subject.

3. The method of claim 1, wherein said sample is selected from the group consisting of:
whole blood or any fraction thereof, erythrocytes, platelets, saliva, septum, tears, and feces.

4. The method of claim 1, wherein said at least one phosphoinositide is selected from the group consisting of: PI4,5P2, PI, PI4P, and PI3,4,5P3.

5. The method of claim 1, wherein said determining comprises contacting said sample obtained or derived from said subject with a phosphoinositide antagonist having specific binding affinity to: PI3P, PI4P, PI5P, PI4,5P2, PI3,4P2, PI3,5P2, or PI3,4,5P3.

6. The method of claim 1, wherein said at least one phosphoinositide is PI4,5P2.

7. The method of claim 6, wherein said antagonist has specific binding affinity to: PI4,5P2.

8. The method of claim 7, wherein said antagonist comprises any one of: an antibody, a phosphoinositide binding protein or a binding domain thereof, a soluble receptor, and any functional fragment thereof.

9. A method for ameliorating or treating a subject afflicted with any one of: an alpha-synuclein related, PD, and any combination thereof, comprising administering to said subject a therapeutically effective amount of a pharmaceutical composition comprising an agent characterized by being capable of modifying any one of: non-steady state levels of PI4,5P2, PI4,5P2-related signaling, and both, thereby treating the subject afflicted with an alpha-synuclein related disease.

10. The method of claim 9, wherein said modifying comprises increasing or decreasing any one of: said non-steady state levels of PI4,5P2 to physiological steady state levels, said PI4,5P2-related signaling, and both, in said subject.

11. The method of claim 9, further comprising a monitoring step proceeding said administering, comprising at least once determining the amount of at least one phosphoinositide in a sample obtain or derived from said administered subject.

12. The method of claim 11, wherein said determining comprises contacting said sample obtained or derived from said subject with a phosphoinositide antagonist having specific binding affinity to PI4,5P2.

13. The method of claim 12, wherein said antagonist comprises any one of: an antibody, a phosphoinositide binding protein, or a binding domain thereof, a soluble receptor, and any functional fragment thereof.

14. The method of claim 12, wherein said antagonist comprises any one of: an antibody, a phosphoinositide binding protein, or a binding domain thereof, a soluble receptor, and any functional fragment thereof.

15. The method of claim 1, wherein said alpha-synuclein related disease is selected from the group consisting of: PD, Lewy body dementia, Alzheimer's disease (AD), multiple system atrophy, NIEMANN-PICK-type A, and any combination thereof.

16. The method of claim 1, wherein said PD comprises PD with dementia (PDD).

17. The method of claim 1, wherein said agent is 3-(4-carbamoylphenyl)-N-(5-cyanopyridin-2-yl)-N-methylpyrazolo[1,5-a]pyridine-5-carboxamide, 5-2-amino-1-(4-morpholinophenyl)-1H-benzo[d]imidazol-6-yl)-N-(2-fluorophenyl)-2-methoxypyridine-3-sulfonamide, or a combination thereof.

18. A kit for diagnosing, prognosing, or both, any one of: an alpha-synuclein related disease, PD, and any combination thereof, in a subject, the kit comprising:
a. at least one phosphoinositide antagonist; and:
b. a baseline control;
c. a calibrating control; or
d. (b) and (c).

19. The kit of claim 18, further comprising instructions for determining an amount of said at least one phosphoinositide, in a sample obtained or derived from a subject.

20. The kit of claim 18, further comprising any one of: an agent characterized by being capable of inhibiting or reducing alpha synuclein activity, pathogenicity, or both, an anti-PD therapeutic agent, and both, in a subject in need thereof, optionally wherein said kit is for any one of: (i) ameliorating or treating a subject diagnosed or prognosed for said alpha-synuclein related disease, said PD, and any combination thereof; and (ii) monitoring any one of: disease progression or regression, responsiveness of a subject to therapy, or both, of any one of: said alpha-synuclein related disease, said PD, and any combination thereof.


SEQUENCE LISTING (12 sequences)

SEQ ID NO: 1 - DNA, 40 bp, synthetic construct
  misc_feature 1..40: note = Synthetic
  source 1..40: mol_type = other DNA
gaatcaccggtgccgccaccatggatgtattcatgaaagg

SEQ ID NO: 2 - DNA, 30 bp, synthetic construct
  misc_feature 1..30: note = Synthetic
  source 1..30: mol_type = other DNA
taactctagattaggcttcaggttcgtagt

SEQ ID NO: 3 - DNA, 18 bp, synthetic construct
  misc_feature 1..18: note = Synthetic
  source 1..18: mol_type = other DNA
ccgttgtcaggcaacgtg

SEQ ID NO: 4 - DNA, 20 bp, synthetic construct
  misc_feature 1..20: note = Synthetic
  source 1..20: mol_type = other DNA
agctgacaggtggtggcaat

SEQ ID NO: 5 - DNA, 21 bp, synthetic construct
  misc_feature 1..21: note = Synthetic
  source 1..21: mol_type = other DNA
tcaccgagctgcaagaactct

SEQ ID NO: 6 - DNA, 18 bp, synthetic construct
  misc_feature 1..18: note = Synthetic
  source 1..18: mol_type = other DNA
cccacaccttgccgatgt

SEQ ID NO: 7 - DNA, 20 bp, synthetic construct
  misc_feature 1..20: note = Synthetic
  source 1..20: mol_type = other DNA
gcagggagcattgcagcagc

SEQ ID NO: 8 - DNA, 21 bp, synthetic construct
  misc_feature 1..21: note = Synthetic
  source 1..21: mol_type = other DNA
ggcttcaggttcgtagtcttg

SEQ ID NO: 9 - DNA, 20 bp, synthetic construct
  misc_feature 1..20: note = Synthetic
  source 1..20: mol_type = other DNA
gctttgatgcactctgccac

SEQ ID NO: 10 - DNA, 20 bp, synthetic construct
  misc_feature 1..20: note = Synthetic
  source 1..20: mol_type = other DNA
agctcattgttcatgctccc

SEQ ID NO: 11 - DNA, 19 bp, synthetic construct
  misc_feature 1..19: note = Synthetic
  source 1..19: mol_type = other DNA
caccatctggtggctgttc

SEQ ID NO: 12 - DNA, 20 bp, synthetic construct
  misc_feature 1..20: note = Synthetic
  source 1..20: mol_type = other DNA
tcactctgtttgcggatgtc